(19) United States
(12) Patent Application Publication
(10) Pub. No.: US 2007/0122798 A1
(43) Pub. Date: May 31, 2007

(54) Methods and tools for screening active rna in cellulo

(76) Inventors: Sylvie Barcellini-Couget, Mougins (FR); Alain Doglio, Saint Andre (FR)

Correspondence Address: NIXON & VANDERHYE, PC, 901 NORTH GLEBE ROAD, 11TH FLOOR, ARLINGTON, VA, 22203, US

(21) Appl. No.: 10/547,489

(22) PCT Filed: Mar. 3, 2004
(86) PCT No.: PCT/FR04/00499

§ 371 Date: Aug. 31, 2005

(30) Foreign Application Priority Data
Mar. 4, 2003 (FR) 03/02633

(51) Int. Cl. C12Q 1/70; C12Q 1/68; C12N 15/867

(52) U.S. Cl. 435/5; 435/6; 435/456

(57) ABSTRACT

The invention relates to methods and compositions for screening active RNA in cellulo. The invention more particularly relates to methods of the preparation and selection of random RNA providing a cell with a desired phenotype, to random RNA banks and the production thereof, as well as to cellular populations which are transformed by said banks. The invention further relates to identified active RNA for research or therapeutic uses, in order to induce a desired phenotype in a cell in vitro, ex vivo or in vivo, more particularly in human cells.

### INTRODUCTION

This invention relates to methods and compositions for screening and selecting active RNA in cellulo. More particularly, it relates to methods for the preparation and production of banks of RNA expression cassettes or banks of cells encoding random RNA, as well as their use for the selection of active RNA capable of producing or altering a cellular phenotype of interest. The invention also relates to the use of active RNA identified for the purpose of discovering new genes (“drug discovery”), for validating the function of genes, for developing pharmacological tools, for diagnosis or for therapeutical purposes, more particularly with the possibility of correcting, by means of RNA, the expression of pathogenic phenotypes in a cell in vitro, ex vivo or in vivo, particularly in human and animal cells.

### SCOPE OF THE INVENTION

The possibility of acting or interfering in a specific way with biological targets can have multiple applications, more particularly in therapeutical, diagnostic, vaccinal or experimental fields. Thus, the capability of regulating the expression of a gene can make it possible to block or to restore an activity in a cell and/or to correct a pathology. The ability to block the expression of genes of pathogens can make it possible to stop their development, etc.

The expression of a gene results from the superposition of several stages including the synthesis of the messenger RNA, the intracellular metabolism of this RNA, the production of the protein, and finally the stability and the activity of this protein. Inhibiting the expression of a gene thus consists of acting on one of these stages. Furthermore, other metabolites, signalling paths or cellular components can be targeted, such as sugars, lipids, nucleosides, etc. In this perspective, RNA appear to be powerful tools capable of acting effectively, specifically and in a discriminating way on most of the stages of gene expression or on biological targets. Indeed, the structural plasticity of RNA generates a diversity of structural motifs capable of binding with most biological targets, and more particularly organic molecules present in cells (RNA, DNA, proteins and various metabolites such as lipids, sugars, etc.). The interactions involved are generally very specific, and this gives the RNA a strong selectivity with regard to its target, and the intracellular accumulation of RNA is neither toxic nor immunogenic. The possibility of developing, selecting and exploiting the potential of RNA would therefore make it possible to consider numerous beneficial therapeutical approaches because they are selective and non-toxic for the organism (Famulok and Verma, 2002). Different strategies have been considered in the prior art for identifying or producing this type of RNA. More particularly, one can cite the SELEX technology, intended for producing and selecting random structural RNA in vitro (Gold, 1995). One can also cite approaches which aim to express banks of antisense RNA or of ribozymes in cells, so as to test their biological activity (WO99/41371, WO99/32618, WO98/32880). Nevertheless, up to the present day, no approach from the prior art has made it possible to produce or select active RNA in cellulo, in optimal conditions and in an active configuration. Thus, the in vitro approaches do not predict properties of the molecules in vivo because they do not make it possible to predict their cellular penetration, their cellular localisation, their stability, their resistance to nucleases, or their biological activity. In the same way, the approaches described in the applications cited above are not directly exploitable for the expression of structural RNA which are active in particular configurations, or for the effective expression of active RNA in cells. In the same way, application No. WO00/58455 proposes approaches for the screening of RNA, but the implementation conditions of which do not allow an effective exploitation of the potential of these agents. Thus, the tools, vectors and structures mentioned in this application involve repeated in vitro selection stages, impose an individual in cellulo selection of the structures, and so do not allow a rapid and simple selection of RNA banks in active configurations in cells.

### SUMMARY OF THE INVENTION

This application makes it possible to overcome the disadvantages of the prior art. This application now provides new methods and new tools for the production, expression and selection of so-called “active” RNA sequences in cells, notably those of mammals.

More particularly, this invention centres on the implementation of specific conditions for preparing and screening active RNA, making it possible to select candidate compounds which are particularly beneficial. More particularly, the invention is based upon the use of particular structures, making it possible for RNA sequences to be expressed and localised within the cell, in an active and stable conformation, and in chosen compartments. The invention is also based upon the design and use of particular expression cassettes which guarantee a high level of expression efficacy in mammalian cells, and having an inducible character. Moreover, the invention describes the structure of improved viral vectors, making it possible to select active RNA effectively and on a large scale, and the production of products which can be used directly for therapeutical applications. The active RNA motifs are thus selected directly in the cell with the help of innovative approaches, and more particularly in accordance with the approach identified by the name “SECAR” (“Selective Enrichment of Cellular Aptamer RNA”).

A first aspect of the invention therefore relates to methods for the in cellulo selection, identification or optimisation of active RNA. The methods of the invention can be adapted for the in cellulo selection of active RNA capable of giving a desired phenotype to a cell, including active RNA capable of altering the activity of a determined biological target. More particularly, the methods of the invention comprise:
1) contacting a bank of nucleic acids comprising a plurality of species of recombinant retrovirus, each species of retrovirus comprising an expression cassette derived from a VA gene of an adenovirus, possibly made inducible, expressing a distinct random structural RNA, or a part of the same, with a population of cells under conditions allowing the infection of said cells by said recombinant retroviruses,
2) selecting cells having the desired phenotype, and
3) identifying the cassette(s) contained in said cell(s) or of the RNA that they express, said
   RNA being capable of giving the desired phenotype to said population of cells.

The insertion of an active motif within a stable RNA structure (VA RNA) makes it possible to design a global molecular entity, identified by the inventors by the term “aptRNA”, made up from one part which is common to all of the aptRNA (the VA shuttle) which serves to stabilise, present and convey the active motif into the cell. This type of intracellular vehicle for aptamers is innovative and makes it possible to considerably improve the intracellular production of RNA aptamers in relation to the techniques from the prior art.

The possibility of inducing the expression of the cassettes during the selection stages makes it possible to directly validate the effect of the active ARN in the cells selected by means of the simple comparison of the cellular phenotypes observed by comparing the repressed and induced states (FIG. 15, panel A). This method therefore considerably simplifies the phenotype screening stages on mammalian cells for the in cellulo identification of active RNA, and for this reason it is a noteworthy improvement of this type of approach in relation to the prior art.

The direct production and selection, in the cells, of active RNA stabilised in accordance with the invention is beneficial because it makes it possible to select molecules which are already in an active intracellular conformation.

As will be described in detail in the text below, stages 1) to 3) can be repeated one or several times from cassettes or active RNA identified in stage 3) in order to improve, over the course of the cycles, the selection and/or the quality of the active RNA, and/or to adapt or to modulate their properties.

Furthermore, in accordance with the application being considered, the bank of starting nucleic acids can be more or less complex and more or less constrained. Thus, it can be a non-constrained random bank, or a bank produced from the sequence of a given target gene or comprising motifs or imposed residues, in accordance with the profile of the desired active RNA. It can also be a bank pre-selected in vitro, more particularly a coding bank of the RNA pre-selected in vitro, for example a bank produced from the sequence of RNA selected in vitro for a specific property (for example their capacity for binding to a target of interest).

In comparison to the existing approaches of the SELEX type, the benefit of the method is to select not a structural RNA motif considered in isolation outside the context of its intracellular expression, but a global molecular entity, the aptRNA, showing an affinity for a pre-identified target and already in a confirmation in accordance with that of its intracellular expression.

Alternatively, at the end of stage 3) of the final cycle that takes place (or possibly of one or more or any intermediary cycle), an additional stage 4) can be implemented so as to confirm the biological activity of the active RNA selected.

Advantageously, the invention therefore proposes an innovative combination of particular genetic elements in accordance with a particular selection methodology, thus forming a global concept of phenotype screening which is integrated and simplified. In comparison to the techniques proposed by the prior art, the tools and methods described in this application offer numerous benefits. The invention thus shows that expression cassettes derived from an adenovirus VA gene can be integrated and expressed in a retroviral vector context. Retroviral transfer is very effective and makes it possible to control the number of copies integrated by cells and the cassettes constructed, derived from VA genes, form integrated cassettes, ie. containing all of the information necessary for an efficient transcription (promoter, Stop signal, structure responsible for the stability of the RNA in the cell, structure responsible for exporting RNA to the cytoplasm, different possibilities for inserting exogenous sequences, and fairly wide tolerance in relation to the promoter). Moreover, the invention shows that the genetic structure of the constructs can be modified in order to control and determine the intracellular localisation of the RNA produced (nucleus, cytoplasm) and to make the expression inducible in a simple way. The combination of the retroviral transfer and a cassette derived from a VA gene therefore makes it possible to obtain high, controlled expression levels in most cell lines and to use identical RNA structures in vitro and in cellulo, thus guaranteeing a more effective selection of active molecules and a faster validation of the active cassettes identified.

The invention is applicable in many fields, and more particularly in order to validate the function of a gene, to search for new targets involved in a cellular function, to find and produce new molecules for diagnosis, pharmacology or therapeutics, etc.

Another aspect of the invention relates to libraries (banks) of random and/or active nucleic acids, possibly contained in cassettes and/or vectors. Thus, one specific object of the invention relates to a bank of nucleic acids characterised in that it comprises a plurality of species of recombinant virus, each species of virus comprising an expression cassette expressing a distinct random (and/or active) RNA under the control of a promoter transcribed by the RNA polymerase III, in particular derived from the sequence of an adenovirus VA gene. The RNA can be a random structural RNA or one with a defined sequence. Preferably, the viruses are recombinant retroviruses.

The subject matter of the invention also includes active RNA expression cassettes comprising a sequence encoding said RNA inserted into a promoter derived from an adenovirus VA gene, said promoter moreover being able to comprise a sequence giving an inducible character and/or a modification which makes it possible to retain the RNA in the nucleus.

The invention also relates to any vector comprising this type of expression cassette as well as recombinant cells containing the same.

The subject matter of the invention also includes any composition comprising an active RNA, a cassette, a vector, or a cell such as those defined above and/or identified or produced by the selection method described in the invention.

The subject matter of the invention also includes pharmaceutical compositions comprising an active RNA, a cassette, a vector or a cell such as those defined above, and a vehicle or excipient which is acceptable on the pharmaceutical level.

The invention also relates to a pharmaceutical composition, characterised in that it comprises an active RNA, said active RNA comprising an active sequence inserted into a modified VA RNA, said modified VA RNA comprising an RNA motif ensuring its localisation in the nucleus of the cell and/or a sequence which provides an inducible character.

The invention also relates to a pharmaceutical composition, characterised in that it comprises the active RNA sequence or the active motif identified within the active RNA. This active RNA sequence or this isolated active RNA motif can, moreover, be chemically modified subsequently, for example in a way that improves its stability in a solution.

The invention also relates to tools, constructs, lines, etc. which are useful for the production of the compositions defined above, more particularly modified VA genes, modified tRNA genes, modified U6 cassettes, vectors or cells comprising the same, and their applications.

The invention also relates to methods for producing pharmaceutical compositions, comprising (i) the screening of a bank of random RNA, as described above, to obtain an expression cassette of an active RNA, and (ii) the conditioning of the cassette or the active RNA sequence in any pharmaceutically acceptable excipient or vehicle.

The invention is useful for the identification, production, expression and/or the selection of any active RNA on cells, more particularly mammalian cells. It makes it possible to prepare active compounds in different situations, more particularly for the production of therapeutical agents, in particular those which are anti-infectious, anti-cancerous, acting on the cell's metabolism, acting on the cell's differenciation process, and the cell's growth capacity, etc. As indicated above, the invention offers numerous benefits in relation to the prior art, and more particularly makes possible the direct, rapid and simple selection of RNA molecules active in the cells.

### LEGEND TO THE FIGURES

FIG. 1 describes the type 2 adenovirus VA1 gene as well as its transcription product. FIG. 1A represents the nucleotide sequences of the VA1-RNA gene (transcribed region) (SEQ ID NO: 1). The nucleotides shown in bold print, from 13 to 24 and from 59 to 68, respectively representing box A and box B, are the elements necessary for the transcription of the VA1 gene by the type III RNA polymerase. Nucleotides 157 to 160 (TTTT), shown in bold print, represent the polymerase III transcription stop signal. Nucleotides 93 to 118, underlined, corresponding to the loop IV sequence, are deleted in the VAΔIV structure (SEQ ID NO: 2). FIG. 1B represents the secondary structure of the type 2 Adenovirus VA1 RNA obtained using Mac DNASIS Pro V3.6 software. The part deleted in the VAΔIV RNA is indicated by two arrows (nucleotides 93 to 118). Nucleotide 120 is mutated in the VAΔIV structure (C is mutated in T).

FIG. 2 represents the comparative study of the production and cellular localisation of the VA1, VAΔIVSrf and VATAR* RNA. FIG. 2A: TAR* sequence (SEQ ID NO: 23) inserted in the SrfI restriction site of the VAΔIVSrf gene so as to generate the VATAR* gene. The TAR* oligonucleotide is derived from the TAR sequence of the Human Immunodeficiency Virus (HIV-1) (Yamamoto et al., 2000). FIG. 2B: Analysis of the expression of the VA1, VAΔIVSrf and VATAR* RNA by Northern Blot. The different RNA are extracted from cells 293, 48 hrs after transfection of these cells by plasmids containing the VA1, VAΔIVSrf and VATA* genes. FIG. 2C: Study of the cellular localisation of the VA1, VAΔIVSrf and VATAR* RNA in human cells 293. The RNA are visualised by hybridisation in situ, 48 hrs after transfection of these cells by plasmids containing the VA1, VAΔIVSrf and VATAR* genes.

FIG. 3 represents the promoter of the U6 snRNA human gene (Genebank access: X07425). The elements required for the recruitment and the activity of type III RNA polymerase are the DSE (Distal Sequence Element: nucleotides −221 to −216), the PSE (Proximal Sequence Element: nucleotides −64 to −46) and the TATA box (TATA Box: nucleotides −31 to −24). At the +1 transcription site is located the Sal I restriction site (GTCGAC), the first nucleotide transcribed being the first nucleotide of this site (G).

FIG. 4 represents the secondary structure of the VAΔIV RNA obtained using Mac DNASIS Pro V3.6 software.

FIG. 5 represents the secondary structure of the VAΔIVSrf RNA obtained using Mac DNASIS Pro V3.6 software. The SrfI cloning site, which allows the insertion of exogenous sequences into the VAΔIV Srf RNA, is indicated by an arrow.

FIG. 6 describes the characteristics of the nVAΔIVSrf RNA (nuclear localisation). FIG. 6A: Sequence of the gene encoding nVAΔIVSrf RNA (SEQ ID NO: 4). The nucleotides shown in bold print, from 13 to 24 and from 59 to 68, representing respectively box A and box B, are the elements necessary for the transcription of the VA1 gene by the type III RNA polymerase. Nucleotides 138 to 141 (TTTT), shown in bold print, represent the polymerase III transcription stop signal. The Srf I restriction site (93 to 100) appears underlined. Nucleotides 120, 121 and 122 written in lowercase represent the mutations introduced into the VAΔIVSrf RNA in order to alter the terminal helix and to give this RNA a nuclear localisation. FIG. 6B: Diagram showing the secondary structure of the nVAΔIVSrf RNA obtained using DNASIS Pro V3.6 software. The SrfI cloning site, which allows the insertion of exogenous sequences into the nVAΔIV Srf RNA, is indicated by an arrow. FIG. 6C: Study of the cellular localisation of the nVAΔIVSrf RNA. The RNA are visualised by hybridisation in situ, 48 hrs after transfection of the 293 cells by a plasmid containing the VAΔIVSrf gene.

FIG. 7 shows the different inducible VA genes (VAi): their sequences, the sequences of the different DNA oligonucleotides which were used for their construction and the study of their level of cellular expression. FIG. 7A: Sequences of the VAiO (SEQ ID NO: 15), OVAi (SEQ ID NO: 16) and OVAiO (SEQ ID NO: 17) genes. The numbering of the sequences starts from transcription point +1; the sequences situated upstream are numbered negatively. The Box A and B sequences, and the transcription stop signal, are shown in bold print. The Tet01 operating sequence is underlined; the SrfI cloning site is shown in italics. FIG. 7B: Sequences of the oligonucleotides having been used for the construction of the VAi genes by primer elongation reaction or by chain polymerisation reaction. The Box A and B sequences and the transcription stop signal are shown in bold print. The Tet01 operating sequences are underlined; the SrfI and PvuII cloning sites are shown in italics. The complementary regions between the VAi up oligonucleotides (SEQ ID NO: 18) and VAi down (SEQ ID NO: 19) appear in lowercase. The hybridisation zones of oligonucleotides Vai PvuII (VAi PvuII 5′ (SEQ ID NO: 20), VAi PvuII 3′ (SEQ ID NO: 22) or OVAi PVuII 5′ (SEQ ID NO: 21)) with the coding regions of the Vai genes are also shown in lowercase. FIG. 7C: Analysis of the expression of the VAi RNA in Hela T-Rex cells following induction with doxycycline (Dox). The different VAi genes (OVAi, VaiO and OVAiO) were cloned in the NheI site of the pBabe plasmid (see FIG. 10). The cells of the 293GP encapsidation line were used for the production of the BabeVAi recombinant retroviruses. The Hela T-Rex cells (Invitrogen ref: R714-07), which express the TetR repressor, were than transduced by the different babeVAi recombinant retroviruses. After selecting cells transduced with puromycin (pBabe selection gene), the different VAi genes were activated by adding Doxycycline (Dox: 1.5 μg/ml). The RNA were extracted at the times indicated, and the expression of the VAi RNA was analysed by Northern blot.

FIG. 8 represents the secondary structure of the VAiO RNA obtained using Mac DNASIS Pro V3.6 software. The region corresponding to the tet01 sequence is defined by arrows.

FIG. 9 represents the secondary structure of the h9U6, h20U6 and nh20U6 RNA, obtained using the Mac DNASIS Pro V3.6 programme. The RNA schematised on this figure represent the non-varying base structures of these RNA (shuttle part). The active motif of the RNA is cloned in the Sfr I site indicated by an arrow.

FIG. 10 represents the pBabe retroviral vector (Morgenstern and Land, 1990). The Nhe I site corresponds to the cloning site of the different expression cassettes of the active RNA.

FIG. 11 shows the relationship which can exist between the expression level of an active RNA and its biological activity. As an example, the active RNA used here is the TAR* aptamer described in FIG. 2B. This TAR* motif corresponds to an RNA motif capable of repressing the replication of the human immunodeficiency virus (HIV). This TAR* motif was cloned on the Sfr I site of the VAΔIVSfr cassette. The VATAR* structure was inserted in the NheI cloning site of the pBabe retroviral vector (see FIG. 10). The cells of the 293GP encapsidation line were used for the production of the Babe and Babe/VATAR* recombinant retroviruses. The anti-HIV efficacy of VATAR* was measured using P4 indicating cells which make up a system currently used for studying the multiplication of HIV (Charneau et al., 1994). The P4 cells were transduced by the Babe and Babe/VATAR* retroviruses and selected by adding puromycin. The expression level of the VATAR* RNA was analysed by Northern-blot within the selected population of P4 cells (parental population), and also in 2 independent cellular clones (clones 1 and 4) isolated from this population. The infection of these different cellular systems with HIV-1 demonstrates that the clone 1 expressing a high level of VATAR* RNA resists the multiplication of HIV more effectively. The rate of infection with HIV-1 is estimated by measuring the activity of the β-galactosidase producing gene contained in the P4 cells, and the results are shown as a percentage of the maximum infection.

FIG. 12 shows an application of the method for selecting active RNA from a library of random RNA expression cassettes cloned in the pBabe retroviral vector. In this application, the active RNA were selected for their capability to make Hela cells resistant to the apoptosis induced by Staurosporine. FIG. 12A: The different synthesis stages of the library of random sequences. The library of single-stranded DNA oligonucleotides includes from 5′ to 3′: 8 underlined fixed bases (run A), 26 random bases 26, then 8 underlined fixed bases (run B). The run A oligonucleotide, complementary to Run B, is hydridised with the library of single-stranded DNA oligonucleotides and elongated by Kleenow fragment DNA polymerase. This neosynthesized double-stranded DNA is called random double-stranded DNA library. FIG. 12 B: Library of random VA Babe vectors. The library of random VA Babe recombinant retroviral vectors is generated by cloning the random double-stranded DNA library in the Srf I site of the VAΔIV Srf gene. This library is therefore made up from a collection of VAΔIV Srf RNA expression cassettes each containing a random motif and cloned in the pBabe retroviral vector. FIG. 12 C: Method for selecting Hela cell clones resistant to staurosporine. The library of random VA Babe vectors is transfected in the encapsidation cells of the 293GP line in order to produce the library of random VA retroviruses. This retroviral library is used in order to transduce the library of random cassettes in the cells of the Hela line in such a way that each cell transduced contains on average just one random VA cassette. After selecting the transduced cells, the cellular apoptosis is induced by adding staurosporine (0.8 μM-6 hrs). The cells which are resistant to staurosporine are amplified in the form of a cellular clone, and the expression cassettes of the active RNA present in each of the clones are analysed. At this stage, 12 cassettes were selected, and their anti-apoptotic activity was studied (FIG. 12D). FIG. 12 D: Validation of the anti-apoptotic activity of the RNA identified by the SECAR selection method. The active RNA expression cassettes selected (in FIG. 12C) were cloned in the pBabe plasmid. These recombinant plasmids were used to produce recombinant retroviruses by the transfection of the cells from the 293GP encapsidation line. Two different cellular lines (Hela and Jurkat) were transduced independently with the different retroviral supernatants (clone 2, 5, 9, 1 1, 13, 14, 15, 16, C, J, L and N) and selected by puromycin. The total cellular RNA coming from these different populations of transduced cells were extracted, then analysed by Northern blot. FIG. 12 E: Individual validation of the anti-apoptotic activity of the RNA identified by the SECAR method. The populations of Hela and Jurkat cells expressing the active RNA selected in FIG. 12C were subjected to tests for resistance to staurosporine (0.8 μM for 6 hours). Hela Cells: The results shown in the histogram reflect the number of cells which are resistant to staurosporine (arbitrary unit). Jurkat Cells: The results shown in the histogram correspond to the percentage of cells which are resistant to staurosporine (measurement of the mitochondrial membrane permeability).

FIG. 13 illustrates the method of the invention with a constrained library pre-selected in vitro (iv SECAR). Use of inducible expression cassettes. FIG. 13 A: Construction of a library of random VAiO transcription cassettes. Stage 1: the Sense bank and iv Antisense bank oligonucleotides are hybridised then elongated by the Kleenow fragment DNA polymerase in order to generate the random VAiO fragment library. Stage 2: the library of random VAiO expression cassettes is obtained by chain polymerisation reaction by using a Taq DNA polymerase, using as a matrix the library of random VAiO fragments and the VAi5′ sense and Bank VAi end antisense oligonucleotides. Stage 3: the library of random VAiO T7 transcription cassettes is obtained by chain polymerisation reaction by using a Taq DNA polymerase, using as a matrix the library of random VAiO transcription cassettes and the 5′ VApT7 oligonucleotides and the 3′ VA end oligonucleotides. FIG. 13 B: Production of RNA derived from the VA gene: VAΔIV Srf RNA, VA TAR* RNA, random VAiO RNA library. The RNA are synthesized in vitro by using the T7 bacteriophage DNA polymerase, and are then visualised on agarose gel after colouring with Ethidium Bromide (200 ng per lead). FIG. 13 C: Obtaining of transcription cassettes derived from the VA gene from RNA synthesised in vitro. An RT-PCR reaction is brought about from different RNA substrates synthesised in vitro by using VapT7 and VA end oligonucleotides. The transcription cassettes corresponding to different VA RNA were obtained: VAΔIV Srf expression cassette obtained from VAΔIV Srf RNA; VATAR* expression cassette obtained from VATAR* RNA; random VAiO expression cassette obtained from random VAiO RNA. In the same way, an equimolecular mixture of the three types of RNA (VAΔIV Srf, VATAR* and random VAiO) makes is possible to obtain an equimolecular mixture of the corresponding cassettes. The RT-PCR reaction products are visualised on 2% agarose gel after colouring with Ethidium Bromide.

FIG. 14 provides an example of the structure of the U6/VA hybrid gene. The VAiO gene is put here under the control of the promoter of the U6 murine gene: gene mU6/VAiO. The first stage consisted of introducing the promoter of the U6 murine gene to the NheI site of the pBabe plasmid. The U6 murine gene (mU6) promoter was obtained by the chain polymerisation reaction using the mU6 primers upstream and the mU6 primers downstream. In the mU6 primer downstream, four bases were added (in italics, bold and underlined). By adding these bases, it was possible to generate the PmeI site indicated in the mU6/VAiO pBabe structure. In the mU6 primer downstream, the four bases AAAC underlined correspond to the last four bases of the U6 promoter and to the first four bases of the Pme I restriction site (GTTTAAAC). In the chain polymerisation reaction, the matrix used corresponds to the genomic DNA of murine cells. The product of this chain polymerisation reaction was purified, then ligated in the pBabe plasmid digested by Nhe I. The ligation product is called pBabe mU6.

The second stage consisted of inserting the VAiO gene into the PmeI site of the mU6 pBabe plasmid. The VAiO gene was produced by a chain polymerisation reaction using the start VAiO primers and NheI End VAiO primers, the matrix being the VAiO pBabe plasmid. The NheI End VAiO primer contains the NheI restriction site shown in bold print and underlined. The product of this reaction was purified and then ligated in the mU6 pBabe plasmid digested by PmeI. The ligation product is called pBabe mU6/VAiO.

FIG. 15 provides a general description of the strategies for screening active RNA in accordance with the invention. Panel A: Method for selecting inducible active RNA expression cassettes (SECAR): This method makes it possible to select, in cellulo, RNA which are capable of giving a cell a specific phenotype. The phenotype screening shown in this panel is implemented by using inducible expression cassettes; it is implemented in six stages. Stage 1: The library of random RNA expression cassettes is transferred onto the cells of interest via a retroviral vector. The transfer is thus optimised: it is adapted to any cellular type and makes it possible to control the number of cassettes inserted for each cell. Stage 2: The cells having integrated the new genetic material (random RNA expression cassette) are selected by a selection gene present in the retroviral vector. Stage 3: By using inducible expression cassettes, the expression of the cassettes in induced so as to activate the transcription of the random RNA. Stage 4: The selection of the cells which express active RNA and which show the phenotype of interest is carried out. These cells are amplified. Stage 5 (facultative): The active RNA expression cassettes present in the cells selected during this stage can be copied and transferred again into the cells of interest. Stages 1 to 4 can thus be repeated as many times as is necessary. Stage 6: In the cellular clones obtained at the end of stage 4 or 5, the activity of the cassettes is validated by comparing the induced or repressed states. The RNA is validated as an active RNA when the required cellular phenotype is observed in the only condition for which the expression cassette is induced. On the contrary, the RNA is not validated as an active RNA when the required cellular phenotype is observed, no matter what the conditions: induced expression cassette or repressed expression cassette. Panel B: Method for selecting active RNA expression cassettes following previous enrichment with active sequences (ivSECAR for “in vitro Selective Enrichment of Cellular Aptamer RNA”). This method, like the SECAR method, makes possible the in cellulo selection of RNA capable of giving a cell a specific phenotype. In this case, the screening is carried out by using a library of random RNA, enriched in RNA which are capable of binding a given target. The first stages of the screening are carried out in vitro from a bank of random RNA; the RNA used for the in vitro screening have a structure which is identical to those which will be expressed in cellulo in the subsequent stages. Stage 1: From a library of random RNA transcription cassettes, the random RNA library is produced in vitro. The RNA thus synthesized in vitro have the same structural properties as the RNA which are synthesized in cellulo from a bank of random RNA expression cassettes. Stage 2: The random RNA bank is put into contact with the target of interest. Stage 3: The RNA capable of binding the target are retained by an appropriate method. Stage 4: The RNA retained at the end of stage 3 are used to produce a new library of random RNA transcription cassettes, enriched in active sequences. Stages 1 to 4 are thus renewed as wished. At the end of stage 4, a “restricted” library of active RNA expression cassettes is obtained. Stage 5: This restricted library is then used in cellular tests in accordance with the SECAR method described in panel A.

### DETAILED DESCRIPTION OF THE INVENTION

In general, the invention relates to tools and methods for the in cellulo selection of active RNA capable of conferring on a cell a desired phenotype, and the use of these active RNA or of any coding DNA in the experimental or pharmaceutical field, for example. As indicated above, the invention uses, in a particularly beneficial way, the combination of retroviral vectors and cassettes derived from VA genes, which makes possible a simple, efficient and predictive screening, both in vitro and in vivo.

Active RNA

This invention is adapted to the production, expression and/or the selection of any active RNA molecule, ie. RNA molecules capable of interacting with and/or of altering the activity of biological components, and/or of giving to a cell a specific phenotype.

The term active RNA includes, more particularly, structural RNA (such as aptamers) and non-structural RNA, such as antisenses, ribozymes or interfering RNA (siRNA, miRNA or their precursors). The active RNA can be of variable length, typically between 8 in 500 bases, more preferably between 8 and 200, and more particularly between 8 and 150. The active RNA are generally synthesised in the form of single-stranded molecules, even if they can subsequently adopt tri-dimensional structures such as loops, double-stranded regions, helices, etc.

In a specific and preferred embodiment of the invention, the active RNA is a structural RNA, more particularly an aptamer (Famulok and Verma, 2002) (Hermann and Pate, 2000). The aptamers are oligonucleotides capable of specifically binding a target molecule.

In another specific embodiment of the invention, the active RNA is an interfering RNA (siRNA, miRNA or their precursors) (McManus and Sharp, 2002) (Scherr et al., 2003) (Famulok and Verma, 2002) or an antisense RNA.

Indeed, this application is particularly adapted to the design and the screening of RNA, the activity of which requires a particular spatial configuration.

The methods described in this application make it possible to select active RNA from collections or banks of random, general or restricted sequences, which may include a very large number of distinct random sequences. As will be described in greater detail in the text below, the random sequences can be any DNA or RNA molecule comprising at least one unknown sequence element, more precisely of which at least one part of the sequence is random. The selection of active RNA in accordance with the invention is carried out in cellulo, by inserting random sequences into expression cassettes, and under specific conditions. One of the main benefits of in cellulo selection is that the target of the active RNA does not have to be chosen a priori; moreover, the selected active RNA is truly effective under the conditions of cellular use.

Promoter Derived from the VA Gene of an Adenovirus

As indicated above, one feature of the invention resides in the use of particular expression cassettes which make it possible to produce, in cellulo, active RNA, in optimal configurations.

In particular, in a particularly beneficial way, the constructs, compositions and methods of the invention use a promoter derived from the sequence of an adenovirus VA gene.

Several pieces of work, including those of the inventors, have demonstrated the usefulness of the VA1 gene for expressing within the cell ribozymes, aptamers or antisenses, the sequence of which was perfectly defined (Medina and Joshi, 1999) (Barcellini et al., 1998) (Bertrand et al., 1999) (Cagnon et al., 1995) (Gwizdek et al., 2001).

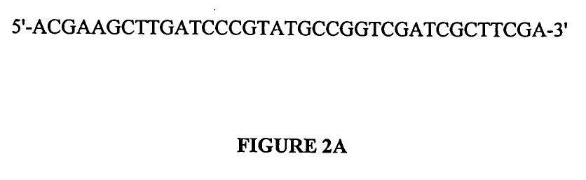

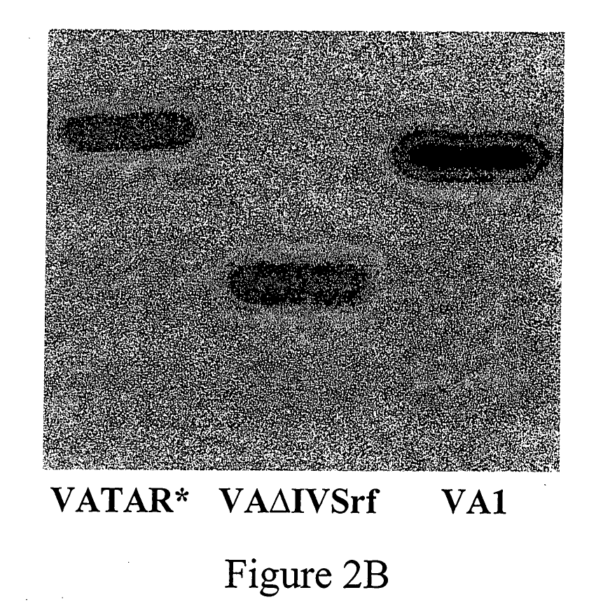

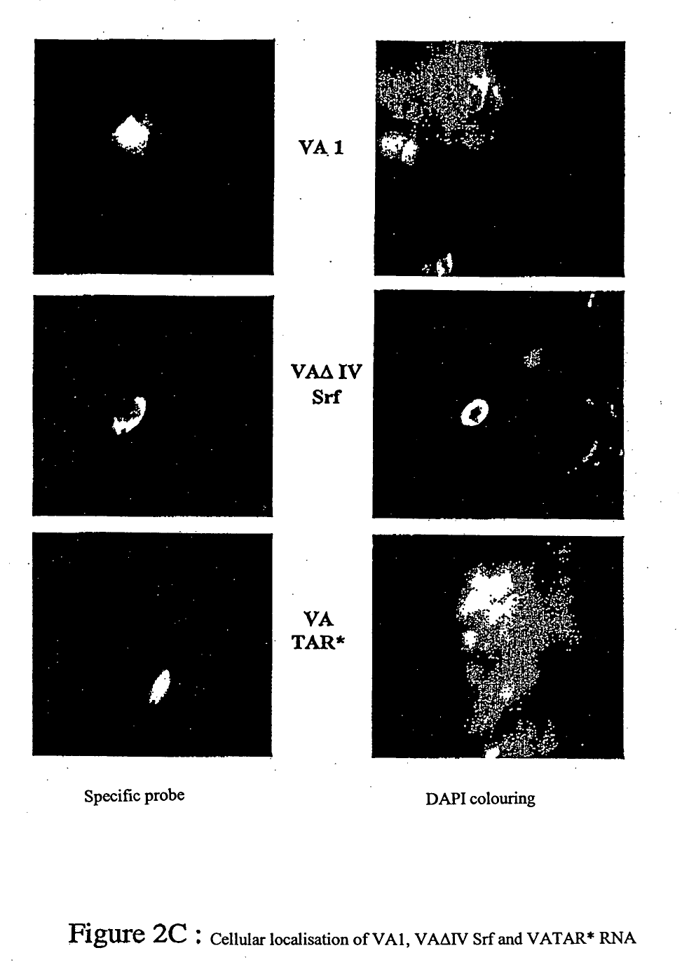

This application demonstrates for the first time the possibility and the efficacy of using this type of cvonstructs for the production and screening of banks of random RNA (or of constrained banks) in cellulo, more particularly in a retroviral context. It also describes different modifications in the VA gene so as to generate improved cassettes which give better control of the expression parameters. The sequences coding for the active RNA can thus be cloned in the cassette, mainly in the central area of the gene. The positioning of the active sequence within the central area does not modify the level of production, or the localisation of the chimeric RNA in the cell (FIG. 2). The use of this type of promoter is particularly beneficial for the expression of structural active RNA, more particularly aptamers.

The adenovirus genome contains two small genes which are transcribed by RNA polymerase III, the VA1 and VA2 genes (Mathews and Shenk, 1991). VA1 RNA, and to a lesser degree, VA2 RNA, are produced in abundance during the adenovirus replication cycle. The interaction between VA1 RNA and a cellular kinase (PKR or p68 kinase) blocks the antiviral effect induced by the interferons, and facilitates the production of new viruses (Mathews and Shenk, 1991).

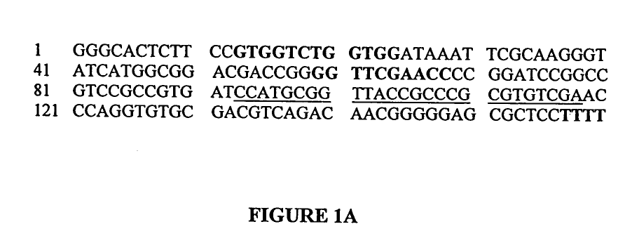

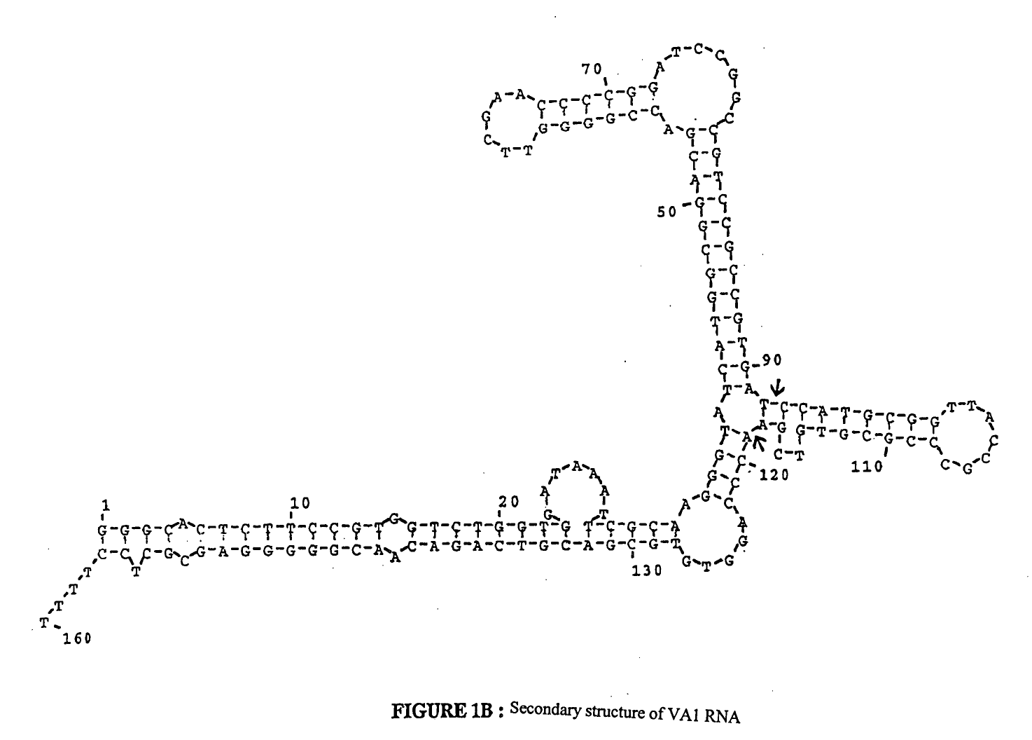

The VA1 adenovirus gene codes for a short RNA (160 nt VA1 RNA) characterised by a rich secondary structure in the double-stranded region. The genetic organisation of this gene transcribed by RNA polymerase III is simple, and comprises a short promoter region in the intragenic position (box A and box B) and a transcription stop signal (see FIG. 1A). The secondary structure of VA1 RNA is well known (FIG. 1B). The sequence of the type 2 adenovirus VA1 gene is represented in FIG. 1A (SEQ ID NO: 1). The corresponding encoded RNA sequence is given below.

| | | | |
|---|---|---|---|
| 1- | GGGCACUCUU | CCGUGGUCUG | GUGGAUAAAU |
| | UCGCAAGGGU | | |
| | AUCAUGGCGG | AGGACCGGGG | UUCGAACCCC |
| | GGAUCCGGCC | | |
| | GUCCGCCGUG | AUCCAUGCGG | UUACCGCCCG |
| | CGUGUCGAAC | | |
| | CCAGGUGUGC | GACGUCAGAC | AACGGGGGAG |

CGCUCCUUUU-160

Boxes A and B correspond to nucleotides 11-24 and 59-68 in bold print, respectively, and the central area containing the loop IV corresponds to nucleotides 93 to 118.

In a first specific embodiment of the invention, the cassette comprises the sequence of an adenovirus VA1 gene deleted of all or a functional part of loop IV, in which the sequence encoding the active RNA is inserted. As indicated above, the central area containing loop IV corresponds to nucleotides 93 to 118 of the VA gene. The sequence of RNA transcribed by expression cassettes in accordance with the invention comprising the sequence of a VA gene deleted from loop IV is given below:

(VAΔIV):

SEQ ID NO:2

| | | | |
|---|---|---|---|
| 1- | GGGCACUCUU | CCGUGGUCUG | GUGGAUAAAU |
| | UCGCAAGGGU | | |
| | AUCAUGGCGG | ACGACCGGGG | UUCGAACCCC |
| | GGAUCCGGCC | | |
| | GUCCGCCGUG | AUAUCCAGGU | GUGCGACGUC |
| | AGACAACGGG | | |
| | GGAGCGCUCC | UUUU-134 | |

The sequence SEQ ID NO: 2 (VAΔIV) is derived from VA1 Ad2 by deleting the central area of 93 to 118. Nucleotide 94 (120 in VA1 ) was mutated such as to create an EcoRV cloning site (in underlined italics). The EcoRV cleavage site is between 92 and 93).

(VAΔIVSrf)

SEQ ID NO:3

| | | | |
|---|---|---|---|
| 1- | GGGCACUCUU | CCGUGGUCUG | GUGGAUAAAU |
| | UCGCAAGGGU | | |
| | AUCAUGGCGG | ACGACCGGGG | UUCGAACCCG |
| | GGAUCCGGCC | | |
| | GUCCGCCGUG | AUGCCCGGGC | AUCCAGGUGU |
| | GCGACGUCAG | | |
| | ACAACGGGGG | AGCGCUCCUU | UU-142 |

The sequence SQ ID NO: 3 (VAΔIVSrf) above is derived from VAΔIV following insertion of the sequence from the SrfI site into the EcoRV site (in underlined italics). The SrfI cleavage site is between 96 and 97.

In another particular embodiment, which can be combined with the preceding one, the terminal double helix of the VA1 gene is altered. This alteration makes it possible to control the cellular localisation of the synthesised RNA (Gwizdek et al., 2001). Thus, when the terminal helix of the VA1 gene is intact, the synthesised RNA is essentially naturally located in the cytoplasm. This localisation is adapted in order to screen active RNA on biological targets essentially present in this cellular compartment. Alternatively, when the terminal helix of the VA1 gene is altered, the synthesised RNA is essentially located in the nucleus. This localisation is adapted in order to screen active RNA on essentially nuclear biological targets. The terminal helix is essentially formed by the matching between the first 20 and the last 20 nucleotides in the sequence of the RNA coded by the VA1 gene. The alteration of the helix can be obtained by different modifications introduced within these sequence elements, more particularly by mutation, deletion and/or insertion, preferably by mutation. When the helix sequence is less than approximately 15 bases, the helix is functionally altered, and the VA1 RNA is retained in the nucleus. Also, when the double helix is mutated such as to introduce a succession of unmatched nucleotides which form an opening within the helix (bubble), the latter is functionally altered, and the RNA is retained in the nucleus.

The sequence of an RNA transcribed by an expression cassette in accordance with the invention comprising the sequence of a VA gene deleted from loop IV and an altered terminal double helix is given below (SEQ ID NO: 4):

| | | | |
|---|---|---|---|
| 1- | GGGCACUCUU | CCGUGGUCUG | GUGGAUAAAU |
| | UCGCAAGGGU | | |
| | AUCAUGGCGG | ACGACCGGGG | UUCGAACCCC |
| | GGAUCCGGCC | | |
| | GUCCGCCGUG | AUGCCCGGGC | AUCCAGGUGU |
| | GCGACGUCAa | | |
| | uaAACGGGGG | AGCGCCCUUU | U-141 |

The sequence SEQ ID NO: 4 (nVAΔIVSrf) is derived from VAΔIVSrf by the mutation of nucleotides 120 to 122 (lowercase) and deletion of nucleotide 136.

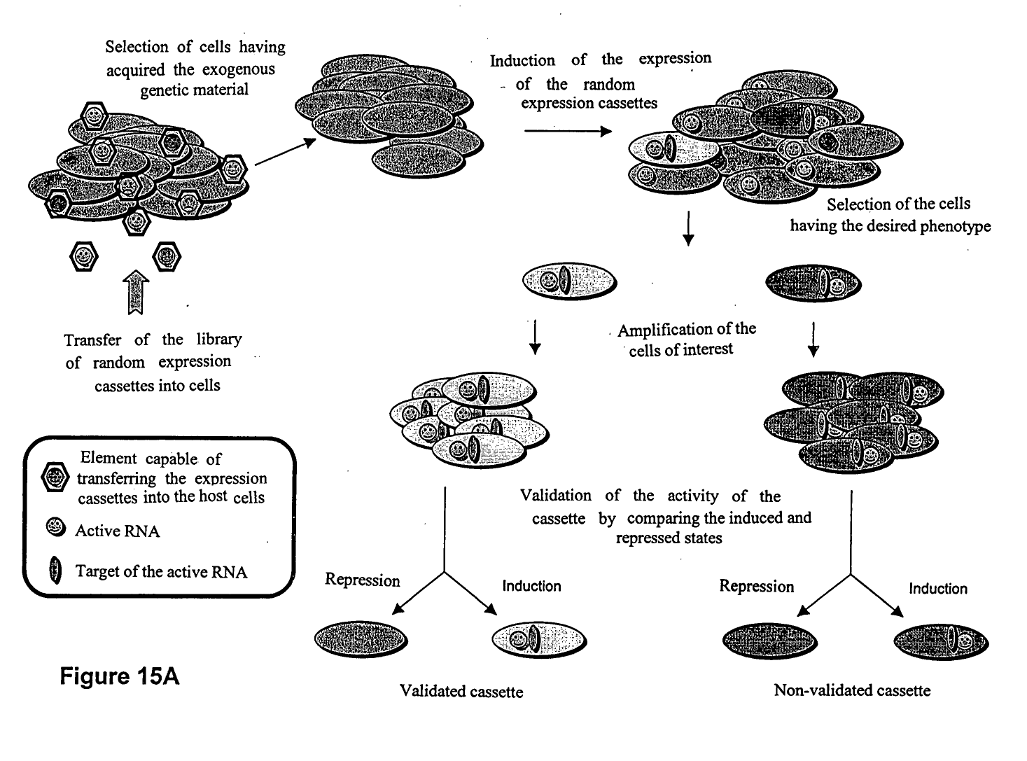

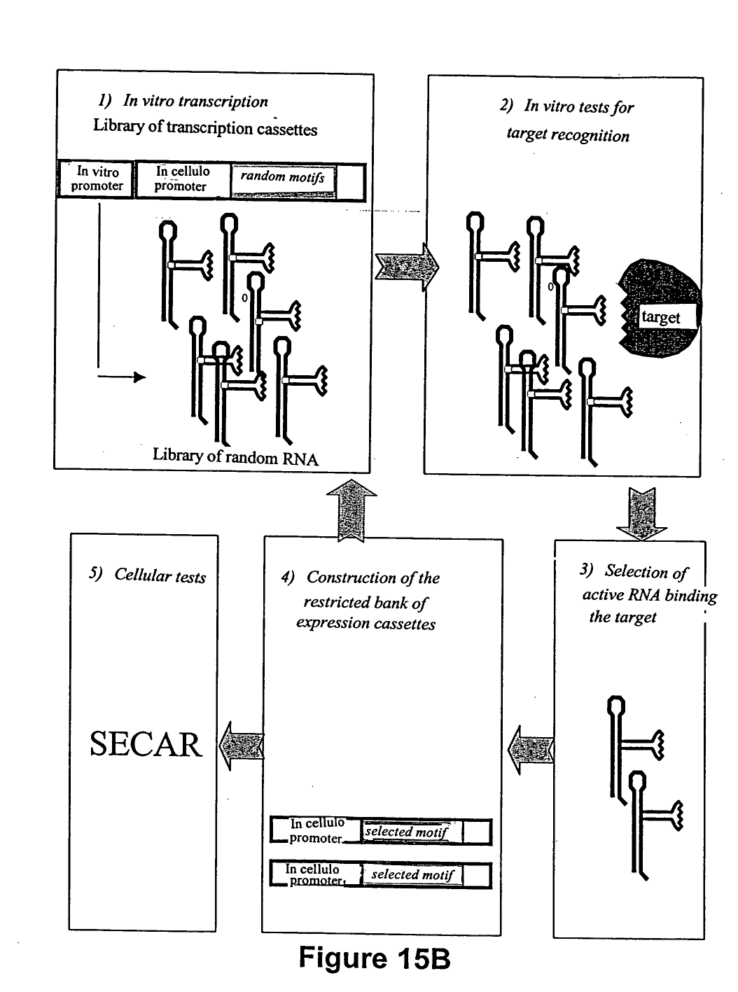

In a particularly preferred embodiment, which can be combined with one or all of the preceding ones, the VA1 gene comprises a sequence conferring an inducible character. Whatever use is made of the active RNA (antisense, ribozyme, aptamer, siRNA, miRNA and their precursors) expressed within a cell, the control of its expression is a determining element. With this objective, this invention shows, for the first time, that the structure of the VA1 genes can be modified such as to make their expression inducible, but at the same time maintaining the characteristics described above (high level of expression, localisation control, the possibility of inserting active sequences in the central area). These results are particularly unexpected, taking into account the intragenic structure of the VA promoter, and make it possible to design expression cassettes which have remarkable properties for the screening of active RNA in cellulo (see FIG. 15A). Thus, the invention proposes and makes it possible, for the first time, to validate in cellulo the activity of cassettes selected by comparing induced and repressed states. This approach makes it possible to reduce the number of false positives and to simplify and to accelerate the identification of RNA of interest.

More particularly, this invention shows that an inducible VA gene can be constructed by inserting one or more sequences which give an inducible character, typically between boxes A and B of the gene and/or upstream of the gene, preferably replacing all or part of the sequences which are normally present. In a case where the sequence is inserted between box A and B, this new sequence is located in a region of the VA1 RNA which is transcribed, and so brings about alterations of the native secondary structure of the VA1 RNA which are likely to make it less stable. In order to correct these alterations, compensating mutations can be introduced within the VA1 sequence so as to correct these structural faults and thus to re-create the natural matching of the VA1 RNA. These optimised cassettes make it possible to control the expression of the active RNA in the cells, and to improve the conditions of a screening or of the use in research or therapy.

In a particular embodiment, the invention therefore relates to a nucleic acid comprising the sequence of an adenovirus VA gene, modified by inserting one or more sequences which provide an inducible character, preferably between boxes A and B and/or upstream of the gene, more preferably replacing all or part of the native sequences.

The sequence giving an inducible character can be any sequence giving sensitivity to a factor acting in trans. It can preferably be a binding site of a transcription factor or a repressor, or any other agent or molecule. In a preferred embodiment, the sequence is one or more operating sequences of a regulated bacterial promoter, for example of the tetracycline promoter.

The promoter of the tet bacterial genes contains two types of operating sequences 01 and 02 which serve as an attachment site for the TetR repressor (Hillen and Berens, 1994). Each of the tet01 and tet02 sites bind a TetR homodimer. Studies have shown that the tet02 attachment site had an affinity to the TetR homodimer which was three to five times greater than tet01 (Hillen and Berns, 1994). The tet01 operating sequences were thus inserted within eucaryotic heterologous promoting sequences so as to obtain a eucaryotic expression system which can be regulated by tetracycline (Gossen and Bujard, 1992). However, this type of modification or application was never considered using the adenovirus VA1 gene, the manipulation of which is particularly delicate taking into account the presence of the promoter in the transcribed sequences.

We inserted one or two tet01 sequences upstream of the VA1 gene so as to make its expression inducible by adding tetracycline. Thus, the sequences located between Boxes A and B or placed upstream of the first nucleotide transcribed from the VA1 gene were replaced by tet01 sequences. In the absence of tetracycline, the expression of the modified VA1 gene can thus be repressed by binding TetR homodimers on the cis-regulating sequences. The addition of tetracycline makes it possible to release the repressor from its attachment on the tet0 sequences and allows the VA1 gene to be expressed normally.

In one particular embodiment, the invention therefore relates to a nucleic acid comprising the sequence of the VA1 gene of an adenovirus, modified by inserting one or more operating sequences serving as an attachment site for the TetR repressor. Preferably, the operating sequence/s is/are inserted between boxes A and B of the sequence of the VA gene and/or placed upstream of the gene, more preferably as a replacement for all or part of the native sequences. The invention also relates to any expression cassette comprising a sequence encoding an active RNA inserted under the control of an inducible VA promoter, (VAi), more particularly by tetracycline. The sequence of specific examples of inducible VA genes constructed in accordance with the invention is represented in the sequences SEQ ID NO: 15 (VaiO), SEQ ID NO: 16 (OVAi) and SEQ ID NO: 17 (OVAiO).

Specific cassettes derived from the sequence of VA genes and allowing expression of random or active RNA are described in the examples which represent specific subject matter of the invention. One can thus cite the cassettes VAΔIVSrf, nVAΔIVSrf, VAi and nVAi.

Furthermore, in a particular embodiment, the expression cassettes of the invention comprise, in addition to the promoter derived from the VA gene of an adenovirus, a second transcriptional promoter, different from the VA promoter. The use of this type of hybrid structure is more particularly interesting when the promoter derived from the VA gene does not function in a particular cellular type. In this case, the RNA which is active and configured in the VA sequence can be expressed under the control of a second promoter which can be of a variety of natures and origins. Advantageously, the second promoter is a promoter transcribed by type III RNA polymerase, and which functions in the cellular type in question, more particularly an extragenic promoter. Downstream of this second promoter, at transcription site +1, the expression cassette derived from the VA gene (VAΔIV, VAΔIV Srf, nVAΔIV, VAi or any other cassette derived from the VA gene) is cloned. In accordance with a preferred embodiment, the U6 gene promoter is chosen as a second promoter which makes possible an active transcription of the VA gene cloned in this way.

A particular part of the subject matter of the invention thus centres on a hybrid expression cassette comprising an expression cassette derived from a VA gene of an adenovirus, possibly made inducible, expressing an active RNA (aptamer, antisense, ribozyme, interfering RNA: Si RNA, mi RNA or their precursors) placed downstream of a second transcriptional promoter. The structure of this type of hybrid cassette is given in detail in the examples (see example A-3) and in the figures.

Promoters Dependent Upon the RNA Polymerase III

In certain specific embodiments of the invention, for example for hybrid cassettes, it can be possible to use other promoters dependent upon RNA Polymerase III for the expression of active RNA. This can be a promoter localised within the transcribed sequences (intragenic) like the tRNA gene promoter. It can also be a promoter localised upstream of the sequences transcribed (extragenic), like the type U6 promoters encoding the small U6 nuclear RNA (snRNAU6). Moreover, the promoter used can be made inducible.

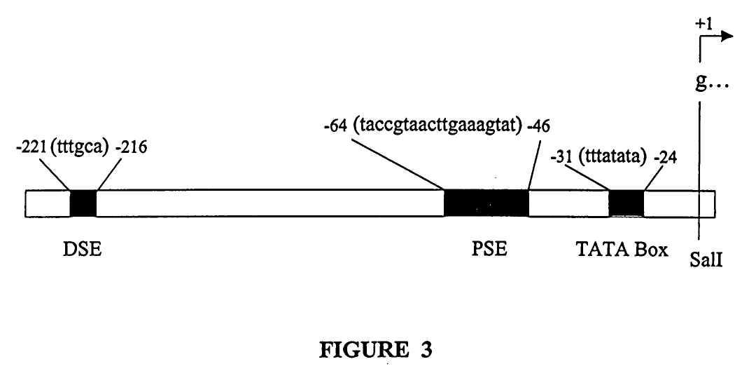

The transcription of the human U6 gene (hU6) generates a small nuclear RNA (RNA snU6) integrated in a ribonucleoproteic complex responsible for the splicing of the RNA (Gmeiner, 2002). The hU6 promoter is placed upstream of the transcribed sequence, and the sequences corresponding to this promoter are therefore not produced in the cell. The main benefit of this is that very few constraints are placed upon the sequence in the transcribed region of the cassette (Bertrand et al., 1997). Indeed, the second transcribed nucleotide corresponds to the first nucleotide produced by the exogenous sequence and the last nucleotides correspond to the stop signal (FIG. 3). It is therefore possible, using this type of cassette, to produce in the cell artificial RNA (antisenses, ribozymes, aptamers, interfering RNA: siRNA, miRNA and their precursors) of which almost all of the transcribed sequence is controlled.

So as to control the stability of the RNA generated, as well as their cellular localisation, different expression cassettes using a U6 promoter have been constructed by the inventors. The cassettes make it possible to express cytoplasmic or nuclear, structural or non-structural, constrained or non-constrained RNA. Modifications have thus been made to the U6 promoter with the aim of facilitating the cloning of active sequences, of controlling the transcribed sequences from transcription point +1, of stabilising the active sequences and controlling the intracellular localisation of these sequences.

In a first embodiment, a sequence serving as the structural base for active RNA is placed downstream of the U6 promoter. This structuring sequence more preferably contains a sequence capable of generating a (short) RNA helix (more or less stable, and more or less long), and/or a free edge cloning site and/or a transcription stop signal (for example TTTTT). The sequence which makes it possible to form an ARN helix typically comprises two complementary regions spaced apart by a hinge region. This type of cassette is useful, above all, for the intracellular expression of structural RNA motifs. When the active motif itself is structured in the form of a helix, the structuring sequence preferably comprises a short shuttle motif (typically with 5 to 12 bases). When the active motif is not structured as a helix or very short, the structuring sequence preferably comprises a long shuttle motif, typically comprising from 12 to 20 bases. This motif can be structured so as to form a perfect helix, guaranteeing a cytoplasmic localisation of the RNA produced, or else structured so as to form a disturbed helix so as to obtain a nuclear localisation of the RNA generated.

In another embodiment which can be combined with the previous one, a sequence forming a short hair-pin structure is placed downstream of the U6 promoter (and should the occasion arise, of the structuring sequence). This sequences forms a stable RNA structure placed at the 3′ end of the active or synthesised random RNA, thus protecting the RNA produced from degradation by RNAses. Downstream of this stabilising structure is located the transcription stop signal of the polymerase III: TTTTT.

In an alternative embodiment, the cassette comprises the U6 promoter to which the sequence encoding active RNA is directly bonded. This type of cassette makes it possible to express RNA, the sequence of which is chosen from transcription point +1.

In accordance with a particular embodiment, the cassette comprises a U6 promoter, like that defined above, and one or more sequences which give an inducible character, as described above. The sequence/s giving the inducible character can be placed, for example, upstream of the promoter sequence.

Specific cassettes derived from the U6 promoter and which make it possible to express random or active RNA are described in the examples which represent particular parts of the subject matter of the invention. One can thus cite the cassettes U6helices, U6Srf, U6Tt, etc.

In other particular embodiments, the promoter transcribed by RNA polymerase III comes from a promoter of the RNAt genes. Moreover, these promoters can be modified so as to give them an inducible character.

Vectors

The expression cassettes in accordance with the invention are typically cloned or included in cloning and/or expression vectors. These vectors can be varied in nature and/or origin, such as plasmids, recombinant viruses, viral vectors, episomes, artificial chromosomes, phages, etc. Preferably, the vectors are of the plasmid, viral or episomic type.

Thus, part of the subject matter of the invention centres on a vector comprising at least one expression cassette as defined above.

When the vector is of the plasmid type, it can come from different known or commercial plasmids. It typically comprises a replication origin compatible with the desired use. It can also comprise a selection gene, and possibly an integration sequence in the chromosome. Examples of plasmids which can be used for the production of vectors of the invention are, more particularly, the pUC, pcDNA, pVV2 plasmids, episomal replication plasmids derived from the Epstein-Bar virus of the OriP type.

In a preferred embodiment, the vector is of the viral type. It can be a recombinant virus, ie. a recombinant viral particle comprising a recombinant viral genome in which at least one cassette, such as those defined above, is inserted. It can also be a viral vector, ie. a genetic structure comprising a recombinant viral genome in which at least one cassette, such as those defined above, is inserted.

In one particularly preferred embodiment, the vector is a retroviral vector or a recombinant retrovirus. Indeed, this application shows that the transfer of a cassette of the invention into the target cells is very effective after the cloning of this cassette in a retroviral vector. More particularly, this application shows, in an unexpected way, that the cassettes derived from VA1 genes can be transduced effectively into the target cells in a retroviral context. In particular, following integration of the retroviral vector containing the cassette into the genome of these cells, the expression level of the chimeric RNA is high, and the properties of the cassette are maintained.

Viruses have been used in order to vectorise nucleic acids in vitro or in vivo. Different approaches have been described for the production of recombinant viruses, typically leading to the production of viruses which are defective for replication, comprising a segment of exogenous nucleic acid encoding a desired product. These viruses have been constructed from retroviruses (MLV, lentivirus, etc.), adenoviruses (Ad5, Ad2, CAV, etc.), AAV, herpes viruses, etc. In each of these approaches, a viral vector is constructed comprising the sequences necessary in cis for the packaging of a nucleic acid in a viral particle, and possibly, additional regulatory or coding sequences.

In the case of the retroviruses, many constructs have been described, in which all or part of the gag, pol and/or env genes are deleted, and in which a nucleic acid of interest is introduced. The latter can be inserted as a replacement for the deleted sequences, or in other regions, such as for example in an LTR. Viral vectors known to skilled persons in the field are notably MFG, pBABE, etc. Typically, a retroviral vector of the invention therefore comprises the LTR terminal regions, the packaging sequence, a selection gene, and the nucleic acid encoding active RNA, in accordance with the invention. This type of viral vector can be constructed from different types of retrovirus, and used in order to produce recombinant viruses by introducing it into a packaging cell line expressing the viral proteins GAG, POL and ENV. This type of cell line has been described in the prior art (PsiCRIP, PA317, Gpenv, 293GP etc.).

A particular part of the subject matter of the invention centres on a retroviral vector which is defective for replication, comprising an LTR sequence, a retroviral packaging sequence, and at least one expression cassette as defined above.

The vectors of the invention can comprise one or more RNA expression cassettes which may be identical or different. Thus, multi-cassette vectors can be constructed, into which cassettes can be inserted, for example in tandem. The possibility of cloning several cassettes in just one vector can involve just one active RNA sequence, or several different active RNA sequences. In the first case, the same cassette is copied several times before being inserted into the vector; in the second case, the different cassettes are placed next to one another and inserted in the vector, or cloned sequentially, in distinct sites of the vector.

The vectors can be constructed by known techniques from molecular biology, more particularly by cloning, ligation, amplification, etc.

As indicated above, the combined use of a retroviral vector and sequences derived from the VA gene of an adenovirus, in accordance with the methods described in this application, make it possible to provide an integrated, simple system which can predict the activity of random RNA, both in vitro and in vivo.

The subject matter of the invention also relates to a composition comprising a vector such as that defined above. The composition can be a pharmaceutical composition, as will be described in greater detail in the text below.

The subject matter of the invention also relates to a composition comprising a plurality of vectors such as those defined above. The composition can be a bank, as will be described in greater detail in the text below.

Bank

In the sense of the invention, the term bank (or library) means a product or a complex composition comprising a plurality or a multitude of components or members which can be present mixed up or in separate compartments. The banks in accordance with the invention typically comprise a plurality of active RNA, or cassettes encoding these active RNA, which can be cloned in vectors, notably plasmids, viral vectors, viruses, and/or in cells. Typically, although not obligatorily, all of the cassettes and/or vectors of a same bank have more or less the same structure, these components differing from one another by their nature (eg. length, origin, type, etc.) and/or their structure (eg. sequence) of the encoded active RNA. Moreover, a bank generally comprises several copies of each component or member. The complexity of the bank can vary to a large extent. A bank can thus be made of two components comprising distinct active RNA expression cassettes, preferably at least 10, or even more preferably at least 20. Typical banks comprise more than 100, 500 or 1000 distinct components, for example as many as 10⁹ or even more. When dealing with banks of random RNA expression cassettes, it is clear that the precise composition (eg. the sequence) of the components of the bank is generally, and on principle, unknown, at least in part. The components of the bank (eg. cassettes, vectors, cells, etc.) can be present in diverse forms, such as in liquid or gel form, or lyophilised, etc. They can be immobilised on a support or by suspension, in soluble form. The bank generally comprises a physical support containing the different members of the bank which can be mixed, at least in part, or separated. The support can thus comprise one or several physically separated compartments, such as flasks, tube, bottles, multi-well plates, etc. The bank can be kept in different forms, notably in liquid suspension or frozen, replicated, etc., in its entirety or in part.

The banks of the invention typically include a plurality of vectors, each comprising an expression cassette of a random RNA as described above, the vectors being at least partially in the form of a mixture. Preferably, the bank comprises at least 50, 100 or 200 vectors encoding a distinct random RNA. It can comprise up to several billion distinct molecular types.

The random sequences can be any DNA or RNA molecule comprising at least one unknown sequence element, more precisely, any DNA or RNA molecule of which at least one part of the sequence is random. Such random nucleic acids can typically comprise a random region, bordered at one or both ends, by a defined sequence region. The random region can comprise, for example, 8 to 50 bases, and the defined region/s can comprise 2 to 10 bases. The random nucleic acid can be a single-stranded RNA produced by chemical synthesis or by amplification or by mutagenesis from any biological matrix, or by the expression of a corresponding DNA. The nucleic acid can also be a DNA, more particularly a random double-stranded DNA encoding a random RNA. This type of random, double-stranded DNA can be prepared from a population of random single-stranded DNAs from synthesis or obtained by amplification and/or mutagenesis techniques, by synthesis of a complementary second strand in accordance with techniques known to experts in the field.

A particular part of the subject matter of the invention resides in a bank of nucleic acids, characterised in that it comprises a plurality of species of recombinant retrovirus, each species of retrovirus comprising an expression cassette derived from a VA1 gene of an adenovirus expressing a distinct random structural RNA.

Another particular object relates to a bank of nucleic acids, characterised in that it comprises a plurality of species of recombinant retrovirus, each species of retrovirus comprising an expression cassette comprising a distinct random RNA under the control of a U6 promoter.

Another particular object relates to a bank of nucleic acids, characterised in that it comprises a plurality of species of recombinant retrovirus, each species of retrovirus comprising an expression cassette comprising a distinct random RNA under the control of a tRNA promoter.

One particular object of the invention resides in a bank of nucleic acids, characterised in that it comprises a plurality of distinct random RNA encoded by distinct expression cassettes derived from a VA1 gene of an adenovirus.

One particular object of the invention resides in a bank of nucleic acids, characterised in that it comprises a plurality of expression cassettes, each comprising a sequence encoding a distinct random structural RNA placed under the control of a promoter transcribed by RNA polymerase III (notably expression cassettes derived from a VA1 gene of an adenovirus), each encoded random structural RNA having the capability of binding a target of interest in vitro.

Another particular object of the invention resides in banks of nucleic acids such as those defined above, in which the expression cassettes comprise an inducible VA promoter.

Another particular object of the invention resides in banks of nucleic acids such as those defined above, in which the expression cassettes comprise a second transcriptional promoter which is distinct, and located upstream of the VA promoter.

In Cellulo Selection

As shown, the invention relates in general to in cellulo methods for selecting active RNA capable of conferring on a cell a desired phenotype from banks of random nucleic acids.

In general, the methods of the invention comprise:
a) the provision of a bank of nucleic acids comprising a plurality of distinct expression cassettes,
   each comprising a nucleic sequence encoding a random RNA placed under the control of a promoter transcribed by the RNA polymerase III,
b) contacting said bank or a part of the same with a population of cells under conditions allowing the transfer of nucleic acids into said cells,
c) the selection of one or more cells having the desired phenotype, and
d) the identification of the cassette or cassettes contained in the selected cell/s, or of the active RNA
   that they express.

In a first embodiment, the bank implemented in stage a) is a general random bank, ie. comprising a plurality of totally random sequences. The use of this type of bank is particularly interesting for the selection of active RNA capable of giving a desired phenotype to a cell, without any a priori knowledge of the biological target in question or of the targeted metabolic pathway.

In another embodiment, the bank implemented in stage a) is a restricted random bank, ie. comprising a plurality of sequences, the random character of which having a certain restriction level. Thus, the restricted bank can be a bank derived from the sequence of a given target gene, comprising a multitude of complementary sequences of one of more regions of this gene. The restricted bank can also be a random bank in which one or more residues, or one or more sequence motifs are imposed within the random region. The restricted bank can also be a bank of random mutants of a given target sequence or a bank encoding RNA pre-selected for a particular property. The use of restricted random banks is particularly interesting for the selection of active RNA capable of altering a determined biological target, or a determined metabolic pathway.

Thus, in one particular embodiment, the bank implemented in stage a) is a restricted random bank encoding random RNA pre-selected for a particular property, for example for their capacity to bind, in vitro, a target of interest (for example a protein, a polypeptide, a peptide, a nucleic acid, a cell, a lipid, etc.) or for their affinity to this target, for one own property, for the presence of a structural motif, or of a specific sequence, etc. In this context, a particular object of the invention relates to a method for the selection, optimisation or identification of active RNA, comprising:
1a) the preparation of a bank of nucleic acids comprising a plurality of distinct expression cassettes comprising a nucleic sequence encoding a random RNA placed under the control of a promoter transcribed by RNA polymerase III, the encoded random RNA sequences, or the whole RNA containing these random sequences having been pre-selected in vitro for their capacity to bind (or for their affinity to) a target of interest,
1b) contacting this bank or a part of this bank with a population of cells under conditions which allow the transfer of nucleic acids into said cells,
2) the selection of one or more cells having the desired phenotype, and
3) the identification of the cassette or cassettes contained in said cell/s, or of the active RNA
   that they express.

The banks can be produced by any technique known to the skilled artisan in the field, more particularly by synthesis, amplification, mutagenesis, etc., or combinations of these methods. It can be a bank of synthetic DNA or of DNA produced by the recombinant or genetic route from artificial or synthetic matrices, such as genomic banks, of RNA from sequences obtained by the SELEX method, or by any mutagenesis or directed evolution technique, etc.

In one particular embodiment, the DNA bank encoding random RNA is prepared by:
synthesis of a single-stranded DNA bank comprising a random region flanked by one or two regions with a defined sequence,
synthesis of a second strand by means of a DNA polymerase and in the presence of a complementary primer of the defined sequence of the first strand, or of a part of the same, in order to produce a bank of double-stranded DNA comprising a random region, and
cloning of the bank of double-stranded DNA in a vector under the control of the chosen promoter.

This method can include an extra stage for the expression and selection in vitro of the RNA encoded by the bank having the capability of interacting with a biological target of interest.

In another particular embodiment, the DNA bank encoding random RNA is prepared from a collection of random RNA sequences by:
reverse transcription in order to produce a bank of single-stranded DNA comprising a random region flanked by one or two regions with a defined sequence,
the synthesis of a second strand by means of a DNA polymerase and in the presence of a complementary primer of the defined sequence of the first strand, or of a part of the same, in order to produce a bank of double-stranded DNA comprising a random region, and
the cloning of the bank of double-stranded DNA in a vector, under the control of the chosen promoter.

In a preferred embodiment, the vector is a viral vector, more particularly retroviral. In this case, the method beneficially also comprises a transfection stage of said vector in a packaging cell line, in order to produce a bank of viruses, more particularly of recombinant retroviruses.

In another particular embodiment, the bank of DNA encodes random RNA pre-selected in vitro (see FIG. 15B). In this embodiment, the method can include the following stages:
Stage A: the in vitro synthesis of a bank of expression cassettes (ds DNA) using the polymerase III system. To this effect, one can, for example, introduce the random active sequence inside of an expression cassette as defined above. The cassettes can be synthesized, for example, from ssDNA oligonucleotides, by means of primer elongation or PCR amplification reactions;
Stage B: the in vitro expression of the bank of cassettes or of a part of the same, producing in vitro a bank of random RNA;
Stage C: the selection in vitro of RNA for their binding capability or for their affinity to a given target; and
Stage D: the production of a restricted bank of expression cassettes comprising RNA expression cassettes selected in this way.

The in vitro expression of the bank of cassettes can be implemented in two phases, one phase for the production of transcription cassettes, and one phase for transcription. For this, a bank of in vitro transcription cassettes can be synthesised from banks of expression cassettes, totally in vitro, by using ssDNA oligonucleotides and by PCR reaction. The dsDNA matrix being produced by the bank of expression cassettes, the 5′ oligonucleotide used in the PCR reaction makes it possible to introduce a promoter adapted to the in vitro transcription (ex: SP6, T7, T3, . . . ). The production of the bank of random RNA can than be brought about by in vitro transcription from the bank of transcription cassettes, using an adapted RNA polymerase (purified protein or preparation which contains the required activity: SP6, T7, T3, . . . ).

The production of the restricted bank of expression cassettes can be implemented in different ways. In a practical way, the active RNA selected (or a part of the same) are used in order to generate the corresponding transcription cassettes, for example by RT-PCR reaction. The new banks of cassettes obtained in this way are used either in order to bring about new method iterations (return to stage B) or for in cellulo tests. The in cellulo tests can involve several identified cassettes, or in a more global manner, this restricted bank of expression cassettes can be used as a starting material for the in cellulo selection (stage 1b).

One particular object of the invention also resides in a method for selecting active RNA, comprising (i) the in vitro synthesis of a bank of expression cassettes (ds DNA) coding for random RNA under the control of a polymerase III promoter, (ii) the in vitro expression of the bank of cassettes or a part of the same, producing in vitro a bank of random RNA, and (iii) the in vitro selection of the RNA for their binding capability or for their affinity to a given target. In an additional facultative stage, the method includes the production of a restricted bank of expression cassettes comprising RNA expression cassettes selected in this way.

During stage b) of the method of the invention, the bank (or a part of the same) is put into contact with a population of cells. The cells used can be varied in nature and origin, and chosen on the basis of the properties required for the active RNA. The method of the invention can thus be implemented, more particularly with a population of cells comprising animal (for example mammalian) cells, birds, fish, amphibians, plants, insects, yeast or bacteria. They are preferably mammalian cells, more particularly those of humans or animals (rodents, cattle, horses, monkeys, etc). The cells can be primary cultures or cell lines. They can be embryonic or somatic pluripotent cells, differenciated or not, proliferative or quiescent, etc. One can name, for example, stem cells, fibroblasts, hepatocytes, epithelial, muscular, renal, nerve or cardiac cells or those belonging to the hematopoietic lineage (lymphocytes B, T, NK, mastocytes, dendritic cells, resident or circulating macrophages, etc), etc. The cells used can furthermore be modified or treated in advance, for example so as to contain a reporter gene system, a marker, etc., or to show a pathological phenotype that one wishes to correct.

The selection method is typically carried out in vitro, in any type of adapted support, such as a phial, flask, multi-well plate, etc. For this purpose, so as to be able to measure or observe, on each cell, the effect of a restricted number of random RNA from the bank, the bank is preferably put in contact with the population of cells in conditions making it possible to transfer a restricted number of cassettes per cell. Indeed, the bank typically being made up from a mixture of distinct components, it is preferred that each cell of the population is modified by a restricted number of these components so as to better appreciate their properties. For this reason, it is not necessary for the components of the bank to be separated from one another, or for the cell population to be divided into supports with several compartments, and this is an important benefit of the invention. In order to show this, when the bank is a bank of viruses, it is preferable to incubate the cells to a weak MOI, typically less than 5, and preferably less than 3, 2 or 1.

The bringing into contact can be performed in the presence of agents which facilitate transfection, such as polymers, cationic lipids, peptides, etc. When the bank comprises viruses, more particularly retroviruses, these types of agent are not generally necessary, taking into account the efficacy of infection.

The cells can be cultivated or conserved for a certain length of time after establishing the contact, before implementing stage c). This length of time can be adjusted by a skilled person in the art according to the desired phenotype, the type of vector, the number of cells, etc. Moreover, following the contacting, it is possible to implement a selection stage for the cells in which one or more cassettes have effectively been transferred. This selection can be implemented by any means known to a skilled person in the art, more particularly by using a marker gene inserted into the vector. Moreover, the cells can also be subjected to particular treatments or conditions, more particularly so as to reveal the phenotype of interest (eg. by adding a substrate, a reagent, or cell lysis, etc.).

The phenotype of interest can be any activity, property, morphology, etc. It can be the expression of an endogenous or exogenous gene, of a marker, of the expression of a surface protein, of a migration, differenciation, growth, resistance property, etc. In one particular embodiment, the desired phenotype is chosen from a capability or incapability relating to growth, apoptosis, differenciation, migration, resistance to a toxic agent, resistance to an infectious agent or metabolic action (eg., the cell has become capable of modifying its metabolic environment). In another embodiment, the desired phenotype is the activity of a determined biological target or of a determined metabolic path. More particularly, one can name the expression or the activity of a protein, for example of an enzyme (eg. kinase, protease, etc.), a transcription factor, etc.

In a first embodiment, the population of cells comprises cells infected by a virus, and the desired phenotype is the resistance to said virus. The virus can be any known virus, such as a hepatitis virus (B, C, delta . . . ), influenza, HIV, the various herpes, the papilloma viruses, etc.

In another embodiment, the population of cells comprises tumoral cells, and the desired phenotype is the loss of tumorigenicity.

In another embodiment, the population of cells comprises non-differenciated embryonic stem cells, and the desired phenotype is the control of their differenciation.

In another embodiment, the population of cells comprises cells capable of acting on a natural metabolic process (for example: blood coagulation, regulation of rates of glucose, lipids, cholesterol . . . ) and the desired phenotype is the control of this metabolic process.

In accordance with another variation, the cell population comprises bacterial cells, and the desired phenotype is the sensitivity to a toxic agent.

In another embodiment, the population of cells comprises cells expressing a determined biological target (eg. a protein, a variant of a protein, a nucleic acid, a lipid, a receptor, etc.), and the desired phenotype is the modification of the activity (including the expression) of this biological target.

The cells expressing the desired phenotype can be selected by a skilled person in the art by any classic technique from biology (morphological modification, survival, expression of a marker, tri-cellular, etc.). Moreover, when the cassette is inducible, the activity of the RNA can be validated directly in cellulo by comparing the induced and the repressed states (FIG. 15A). For this, the cells showing the desired phenotype are selected, possibly amplified, preferably individually, and their phenotype is analysed in parallel under conditions of induction and repression of the expression of the cassette. This extra stage makes it possible to identify the RNA, the activity of which is directly involved in the required phenotype.

Stage d) comprises the identification of the cassette or cassettes contained in the selected cell/s, or of the active RNA that they express. These cassettes or RNA are responsible for the phenotype produced, and so can be used for any application involving reproduction of this phenotype. The cassette, or the RNA can be extracted from the cells and isolated by classic methods of molecular biology (lysis of cells, amplification or hybridisation, etc.). In a preferred embodiment, the sequence of the cassette or cassettes is determined so as to make it possible to produce the corresponding product by the synthetic or recombinant route. Of course, the properties of the cassette or of the RNA can be confirmed in any appropriate system or biological model.

Preferably, when the bank used in stage a) is complex, (ie. comprises a high number of distinct components, for example more than 100), it is preferable to repeat stages b) to d) of the method so as to select the most active agents. In this case, the DNA of the expression cassettes of the selected cells is amplified so as to produce a restricted bank, and stages b)-d) of the method are repeated at least once with said restricted bank. The implementation of several cycles offers several benefits: first of all, it makes it possible to start with very complex banks, used in mixed form. Moreover, it makes it possible to progressively increase the efficacy of the active RNA. Furthermore, it can make it possible to select active RNA having a determined profile, by selecting cassettes on cells or under distinct conditions in accordance with the cycles. Thus, the repetition of cycles can make it possible to control the specificity of an active RNA or, on the contrary, to verify its efficacy on several targets or several cellular types.

One particular part of the subject matter of the invention resides in a method for selecting active RNA capable of giving a cell a desired phenotype, comprising:
a) the provision of a bank of nucleic acids comprising a plurality of vectors comprising distinct expression cassettes each comprising a nucleic sequence encoding a random RNA placed under the control of a promoter transcribed by the RNA polymerase III,
b) contacting said bank, or a part of the same, with a population of cells under conditions allowing the transfer of nucleic acid into said cells,
c) the selection of cells having the desired phenotype,
d) the extraction or amplification of the sequence of cassettes contained in said cells,
e) the cloning of the sequences obtained in d) in a vector in order to generate a restricted bank, and
f) the repetition, at least once, of stages b) and d) with said restricted bank.

In a preferred embodiment, the bank of nucleic acids is a bank encoding random RNA pre-selected in vitro, and/or the vector is a recombinant virus, more preferably a recombinant retrovirus.
In a particularly preferred way, the promoter transcribed by RNA polymerase III is a promoter derived from the sequence of a VA gene of an adenovirus.

In a specific embodiment, the population of cells comprises mammalian cells.

A more specific embodiment of the invention comprises:
a) the provision of a bank of nucleic acids comprising a plurality of species of recombinant retrovirus, each species of retrovirus comprising an expression cassette derived from a VA gene of an adenovirus expressing a distinct random structural RNA,
b) putting said bank, or a part of the same, into contact with a population of mammalian cells under conditions making it possible to infect some of said cells with said recombinant retroviruses,
c) the selection of the cells having the desired phenotype,
d) the extraction or amplification of the sequence of cassettes contained in said cells,
e) the cloning of the sequences obtained in d) in a vector so as to generate a restricted bank, and
f) the repetition, at least once, of stages b) to d) with said restricted bank.

Another particular object of the invention relates to a method for selecting active RNA on a determined biological target, comprising:
a) the provision of a bank of nucleic acids comprising a plurality of vectors comprising distinct expression cassettes, each comprising a nucleic sequence encoding a constrained (or pre-defined) RNA in order to act on said determined target, placed under the control of a promoter transcribed by the RNA polymerase III,
b) contacting said bank, or a part of the same, with a population of cells expressing or containing the biological target, under conditions allowing the transfer of nucleic acid into said cells,
c) the selection of cells having the desired phenotype,
d) the extraction or amplification of the sequence of cassettes contained in said cells, in a facultative manner,
e) the cloning of the sequences obtained in d) in a vector so as to generate a restricted bank, and
f) the repetition, at least once, of stages b) to d) with said restricted bank.

The sequence of the encoded RNA can be derived from the sequence of the biological target (more particularly in the case of the antisenses, RNAi (siRNA, miRNA or their precursors), ribozymes), or else pre-selected so as to interact in a structural way with the biological target (more particularly in the case of aptamers).

Applications for Research in Biotechnology

The active RNA identified, the active sequences identified or the expression cassettes of these active RNA can be used as molecular tools capable of acting in the cell so as to interfere (inhibition, activation) with a biological activity or the expression of a determined phenotype (“target identification”). Used in this way, they are useful products for studying a cellular process and identifying new targets or for exploring the function of a gene in a case where the target is known (“target validation”). Their action in a cell can make it possible to modify the cell in such a way that said cell is endowed with new properties. The cell modified in this way can then be considered as a new tool in biotechnology useable for research purposes or for therapeutical applications.

Pharmaceutical Applications

The active RNA identified and, more generally, the expression cassettes identified, can be used directly as pharmaceutical products. In the context of the invention, the term “pharmaceutical” includes any use in medical, therapeutical, preventative or curative, veterinary, agronomic, diagnostic, cosmetic, etc. fields.

Thus, one aspect of the invention relates to a pharmaceutical composition comprising an expression cassette, a vector or a cell, as defined above, and a pharmaceutically acceptable vehicle or excipient.

Another aspect of the invention relates to a pharmaceutical composition, characterised in that it comprises an active RNA, said active RNA comprising an active sequence inserted into a modified VA RNA, said modified VA RNA possibly comprising an altered terminal helix and/or a sequence giving an inducible character.

The invention also relates to methods for producing pharmaceutical compositions, comprising (i) the screening of a bank of random RNA, as described above, making it possible to obtain an expression cassette for an active RNA, and (ii) the conditioning of the expression cassette or of the active RNA sequence in any pharmaceutically acceptable excipient or vehicle.

In a particular embodiment, the invention relates to a method for producing a pharmaceutical composition for the treatment of an infection by a pathogenic agent in a human patient, comprising (i) the screening of a bank of random RNA, as described above, the population of cells used being infected by the pathogenic agent and the RNA selected for their capability to reduce or block the infectious cycle, making it possible to obtain an expression cassette of an active RNA, and (ii) the conditioning of the expression cassette or of the active RNA sequence in any pharmaceutically acceptable excipient or vehicle.

The invention also relates to the use of an active RNA, an expression cassette, a vector, or a recombinant cell, as defined above, for the preparation of a medicament intended for the implementation of a therapeutical treatment method of the human body. In accordance with the properties of the active RNA, the medicament can be used for the treatment of cancers, infections, neurodegenerative diseases, etc.

The invention also relates to a method for treating a patient, comprising the administration of an effective amount of an active RNA, an active sequence, an expression cassette, a vector or a recombinant cell, as defined above, to a patient. The administration can be made by different methods, more particularly by iv, ip, im, sc, local or general routes, more particularly intratumoral or systemic methods.

Other aspects and advantages of this invention will become apparent when reading the following examples, which must be considered as illustrative and not as limiting.

### EXAMPLES

### .A. Structure of the Expression Cassettes

### A-1. Cassettes Derived from the VA1 Gene of the Adenoviruses

This example describes the structure of cassettes which make possible the expression and intracellular diffusion (in mammalian cells) of active RNA motifs (structural RNA=aptamers, antisenses, ribozymes, RNAi, (siRNA or miRNA) or active motifs). The base of these cassettes is the viral gene VA1 RNA of the type 2 adenovirus. This gene is effectively transcribed by cellular RNA polymerase III. The RNA produced is very structured, its size being 160 bases. The cellular localisation of the RNA is cytoplasmic.

So as to eliminate the physiologically active part of the VAI RNA, the loop IV (which interacts with the p68 kinase protein) was suppressed in this structure, and the EcoRV restriction site was inserted: structure VAΔIV (Barcellini et al., 1998) and Gwizdek et al., 2001).

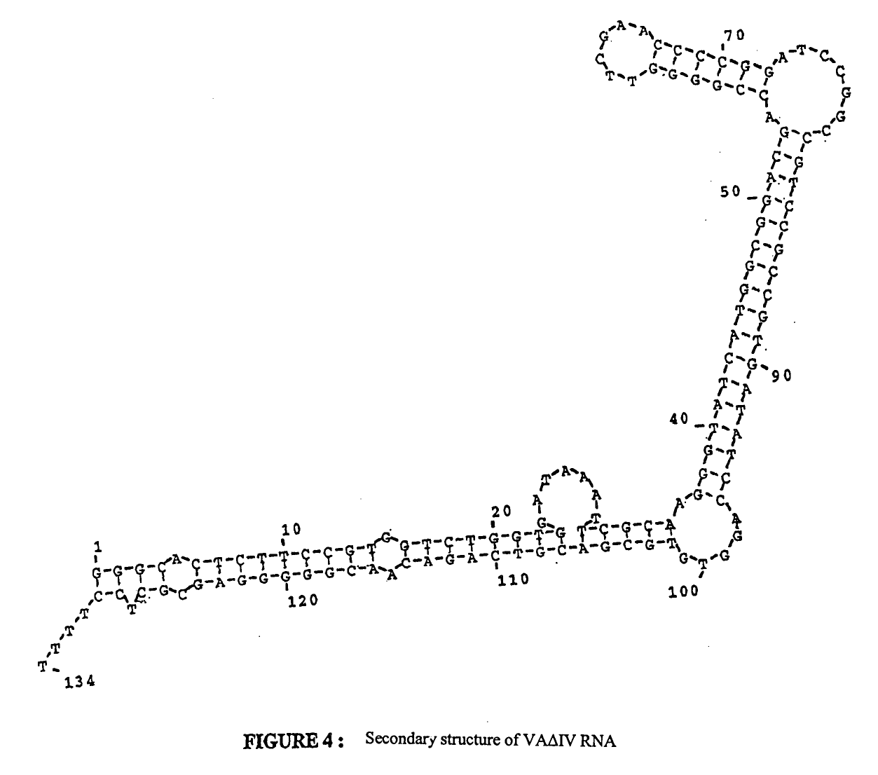

The VAΔIV RNA has a size of 134 bases, it is rich in secondary structures (FIG. 4), and has a cytoplasmic localisation (Barcellini et al., 1998) (Gwizdek et al., 2001). Its sequence is represented by the sequence SEQ ID NO: 2.

### A-1-a. Modification of the VAΔIV Cassette: Cassettes VAΔIV (Cytoplasmic) and nVAΔIV (Nuclear)

Cassette VAΔIVSrf:

This example describes the structure of cassettes which make possible the expression of the active motifs integrated into the VAΔIV RNA, and the localisation of which is mainly cytoplasmic.

In the central area of the VA1 gene, at the deletion of loop IV, the SrfI cloning site, which is further adapted, was introduced, replacing the EcoRV site.

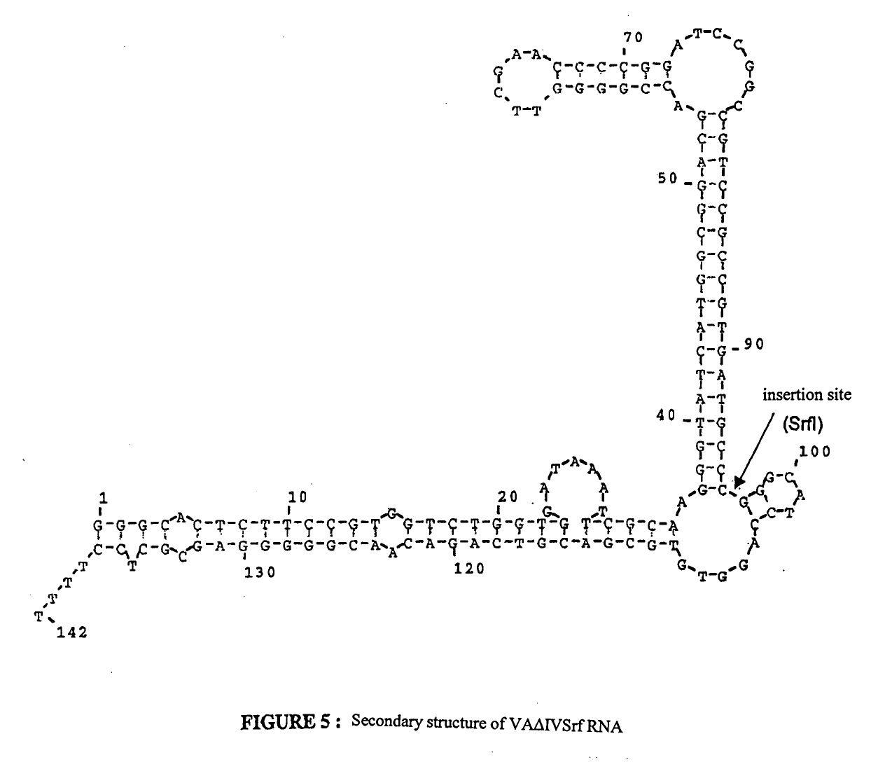

The VAΔIVSrf cassette was generated by inserting the SrfI site in the form of a double-stranded DNA octanucleotide (5′ GCCCGGGC3′) on the inside of the EcoRV restriction site (position 90-96 in VAΔIV). The transcribed RNA has 142 bases (FIG. 5) and a cytoplasmic localisation (FIG. 2C), SEQ ID NO: 3. This RNA make possible the optimised expression of active RNA sequences (FIG. 2B).

Cassette nVAΔIVSrf:

This example describes the structure of cassettes which make it possible to express active RNA sequences integrated into VAΔIVSrf RNA, and the localisation of which remains nuclear.

In native VA1 RNA, the double helix structure containing the 5′ and 3′ ends is the sequence responsible for conveying this RNA from the nucleus to the cytoplasm. This structure is called the terminal helix (Gwizdek et al., 2001). It has been mapped: bases 1 to 20 and bases 136 to 155. If the double helix structure is disturbed, the RNA is retained in the cytoplasm. The 3′ part of the VAΔIV gene has thus been modified so as create a rupture in the terminal double helix of the RNA in accordance with the approach proposed in Gwizdek et al., 2001. For this, the 5′ sequences of the VAΔIV gene have been modified from nucleotide 93, and replaced by the following double-stranded DNA sequence (SEQ ID NO: 5):

5′GCCCGGGCATCCAGGTGTGCGACGTCAATAAACGGGGGAGCGCCC

TTTT3′.

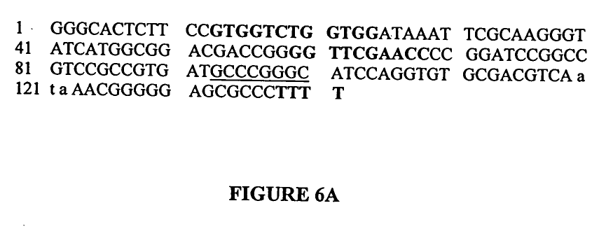

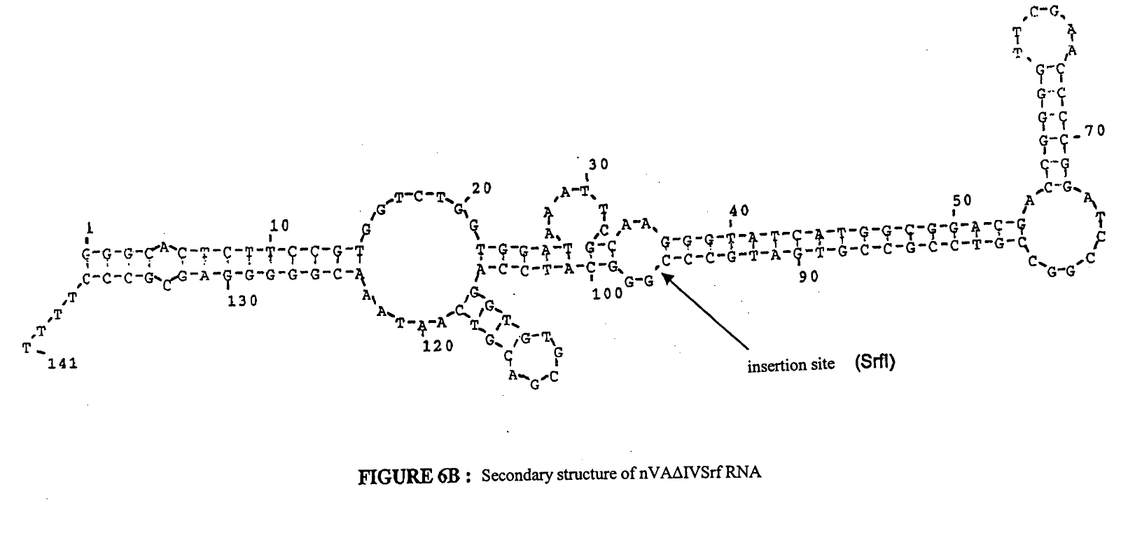

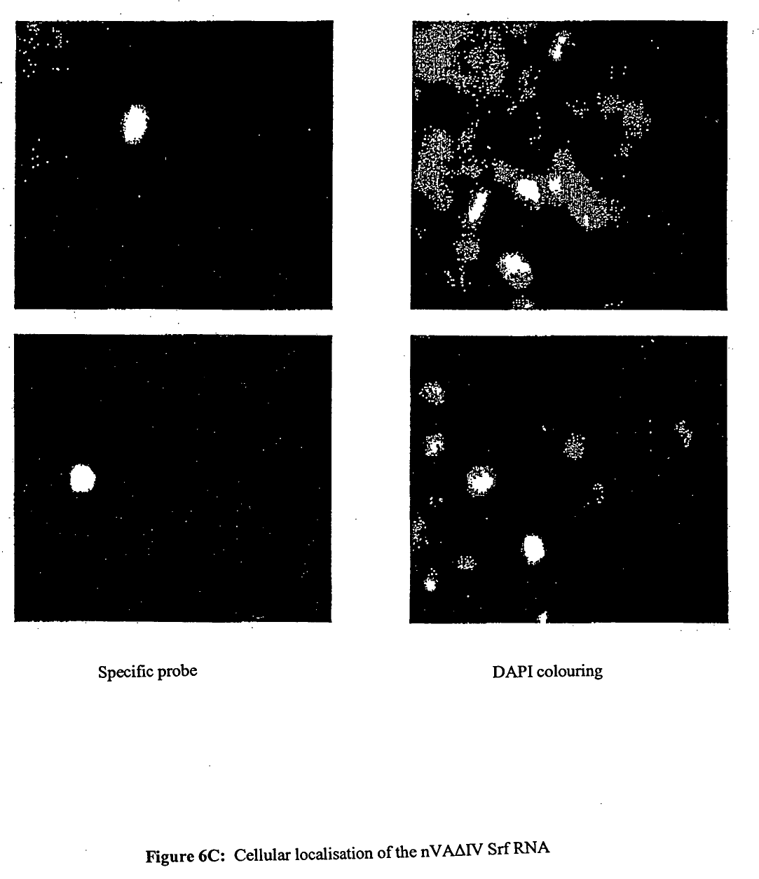

One can see that the SrfI restriction site, in italics and underlined, is directly integrated into this sequence (FIG. 6A). The transcribed RNA, the characteristics of which are shown in FIG. 6B, has 141 bases and a secondary structure close to that of VA1 RNA, its localisation is nuclear (FIG. 6C), SEQ ID NO: 4.

### A-1-b. Inducibility of the VAΔIV Cassette: Cassettes VAi (Cytoplasmic) and nVAi (Nuclear)

VAi Cassettes:

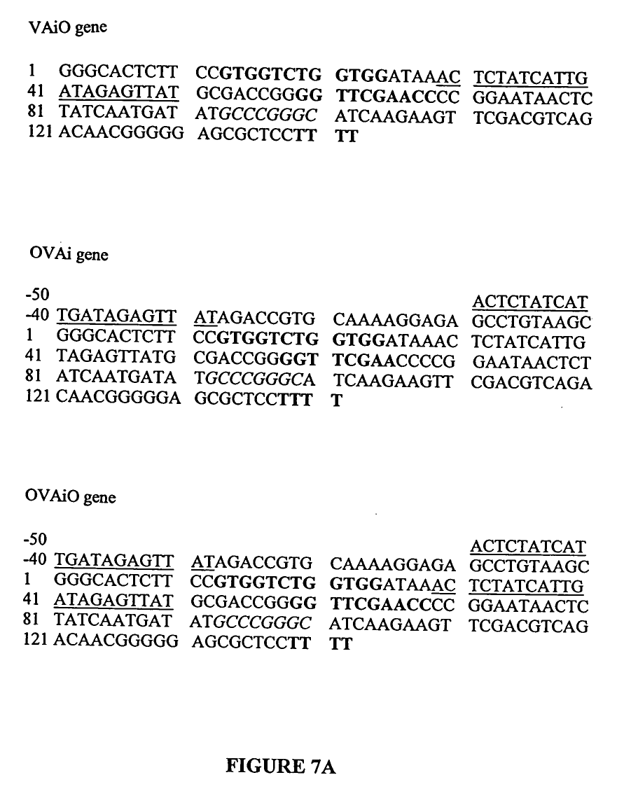

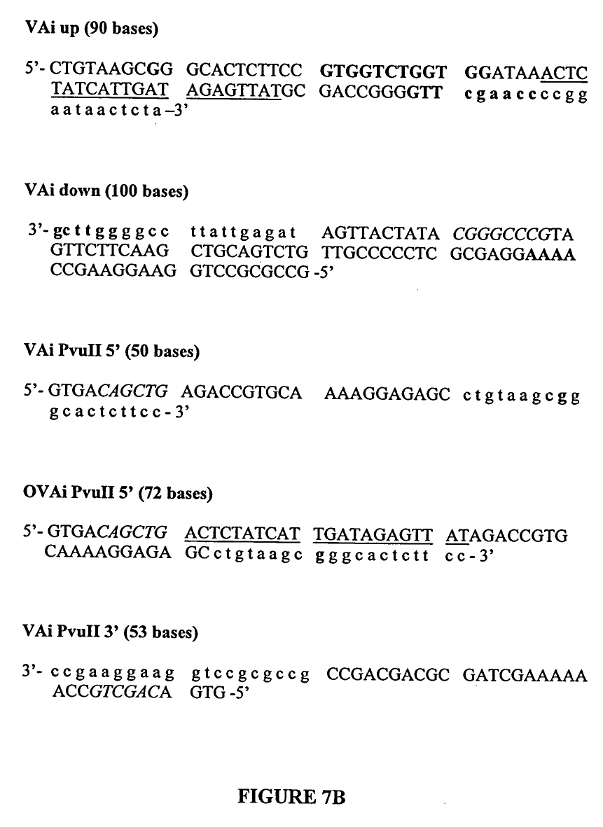

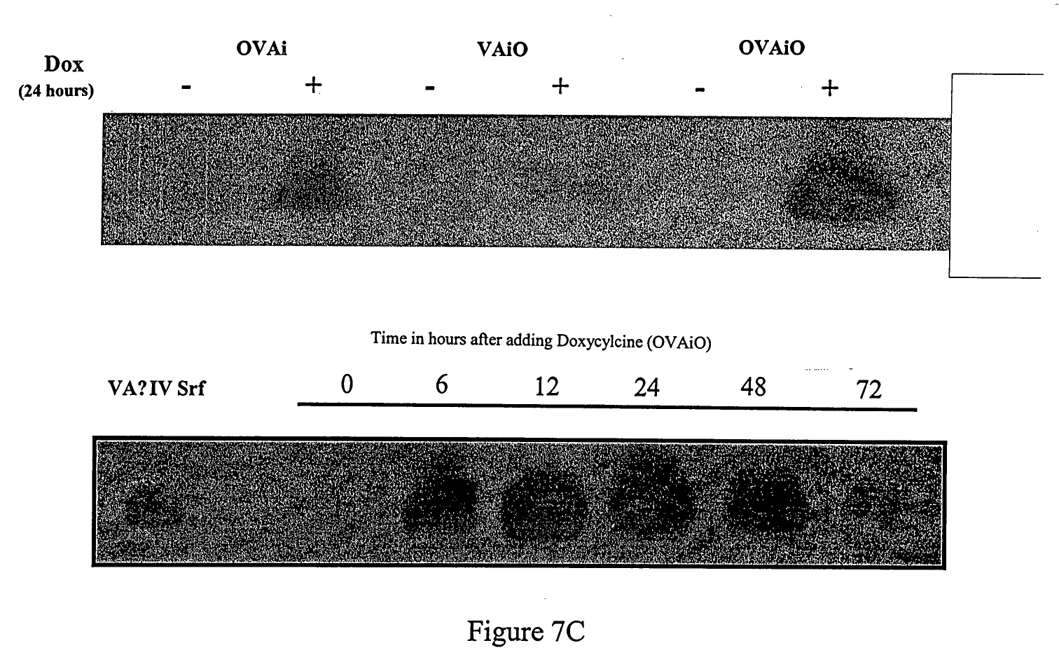

This example describes the structure of cassettes making it possible to express active motifs integrated into an expression cassette which is inducible with tetracycline. Firstly, the VAΔIV gene was modified so as to integrate a tet01 operating sequence between boxes A and B of the VA gene and/or upstream of the initiation site of the transcription of this gene (FIG. 7). The tet01 sequence therefore replaces the native sequences of the VA gene, either between boxes A and B (position 24 to 59 in the VAiO gene), or upstream of the transcribed sequences (position −29 to −50 in the OVAi genes), or at the two positions (extragenic and intragenic: gene OVAiO). Secondly, the SrfI cloning site is located in the central area of this new VA gene. And thirdly, with the aim of recreating a secondary structure close to that of the native VA RNA, sequences partially complementary to the tet01 sequence replace the central sequences of the gene (VAiO and OVAiO). In order to obtain a cytoplasmic VAi RNA, everything is done to conserve the terminal helix responsible for conveying RNA to the cytoplasm. The complete sequence of the OVAi genes (SEQ ID NO: 16), OVAiO (SEQ ID NO: 17) and VAiO (SEQ ID NO: 15) is shown in FIG. 7A.

The VAi cassettes are obtained synthetically using 4 oligonucleotides of single-stranded DNA. The two first single-stranded VAi up and VAi down DNA oligonucleotides (SEQ ID NOs: 18 and 19, FIG. 7B) are used to generate all of the transcribed sequences of the VAi neogene as well as a part of the adjacent 5′ and 3′ sequences. By using a sequence of 20 complementary bases, these oligonucleotides are hybridised, then used in order to generate a double-stranded DNA in the presence of Kleenow fragment DNA polymerase. The two external VAi Pvull 5′ (SEQ ID NO: 20) and VAi PvuII 3′ (SEQ ID NO: 22) oligonucleotides (FIG. 7B) are used so as to add sequences upstream and downstream of the VA gene due to a PCR reaction. The two PVuII restriction sites positioned at the two 5′ and 3′ ends of the VAiO gene make it possible to clone in any DNA vector.

In the same way, the two VAiO2PvuII 5′ and VAiPvulII 3′ oligonucleotides are used in order to add the tet01 sequence upstream of the VAΔIVSrf gene or the VaiO gene so as to generate the OVAi and OVAiO genes respectively.

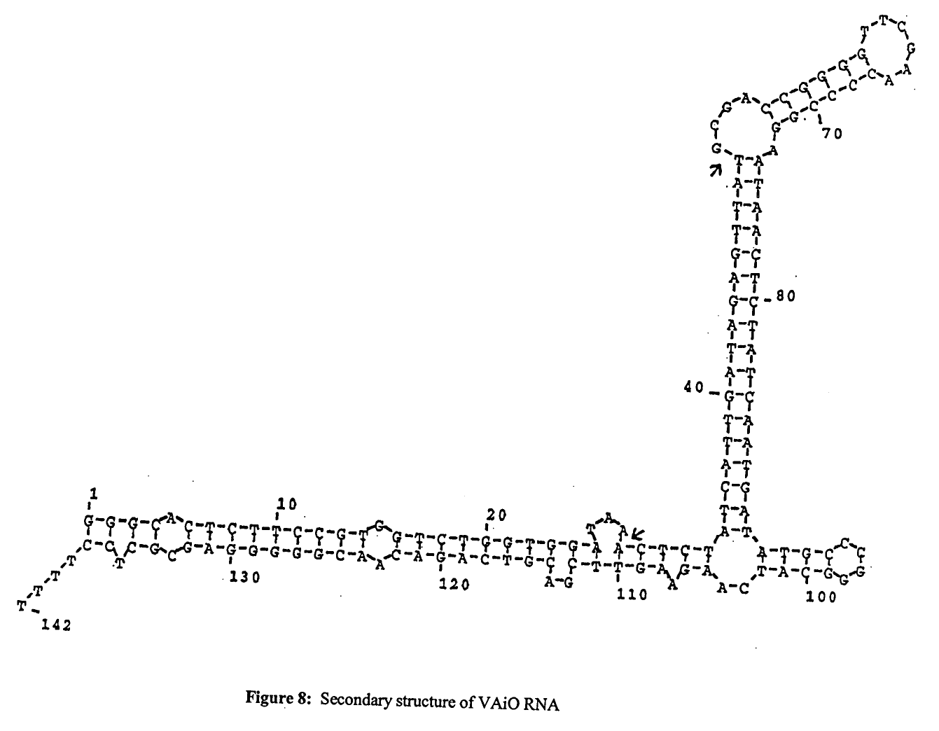

The VAiO transcribed RNA has 142 bases; its secondary structure is shown in FIG. 8.

nVai Cassette:

This example describes the structure of cassettes which make it possible to express active motifs integrated into an expression cassette which is inducible with tetracycline. The localisation of this motif must be mainly nuclear. The principle of retaining this RNA in the nucleus is the same as for the nVAΔIVSrf expression cassette (see above).

The 3′ part of the different VAi genes is therefore modified in the same was as for the nVAΔIVSrf gene so as to create a rupture in the terminal double helix of the RNA.

### A-2. Cassettes Derived from the hU6 Human Gene

The promoting sequence of the human U6 gene is in an extragenic position. The RNA generated therefore corresponds to the sequences that one has chosen to clone downstream of the promoter (from transcription point +1). This type of promoter therefore makes it possible to express entirely synthetic RNA in opposition to the VA system which imposes conservation of the promoting sequences positioned within transcribed sequences.

This example describes several modifications of the U6+1 gene (initially described by (Bertrand et al., 1997)). These modifications make it possible to facilitate the cloning of active sequences, to control the sequences transcribed from transcription point +1, to stabilise the active sequences, and to control the intracellular localisation of these sequences.

The promoter sequences used in the cassettes go from 1 to 266. At transcription site +1 (nu.266), a cloning site has been located with sticky ends: SalI (5′GTCGAC3′) (Bertrand et al., 1997).

### A-2-a. U6 Helix Cassettes (U6h9, U6h20 and nU6h20)

The RNA transcribed by these cassettes have a nuclear or cytoplasmic localisation, depending upon the structures. The aim of these cassettes is the intracellular expression of structural RNA motifs.

A sequence serving as a structural base for the RNA to be inserted is positioned downstream of the U6 promoter. It contains: a sequence capable of generating a short RNA helix (more or less stable, and longer or shorter), a free edge cloning site (SrfI: 5′GCCCGGGC3′) as well as the transcription stop signal TTTTT.

In mammalian cells, the accumulation of double-stranded RNA (more than approximately 40 nucleotides in length) triggers off the production of interferon and the death of cells by apoptosis. However, it is necessary for the RNA shuttle which is supporting the active motif, to have its own, stable structure in the form of a helix. This example describes the structure of three types of shuttle derived from U6: when the active motif is itself structured in the form of a helix, we use a cassette with a short shuttle motif (helix 9: h9U6), when the active motif is not structured as a helix or very short, we use a cassette which makes it possible to express a shuttle formed by the matching of about twenty nucleotides (helix 20 h20U6). h20U6 also makes it possible to modify the terminal stem of the export motif shuttle and to generate a nuclear localisation shuttle (nh20U6).

Different fragments of double-stranded DNA are inserted on the inside of the SalI restriction site using compatible SalI ends.

h9U6:

(SEQ ID NO:6)

5′TCGAGCCCGGGCTCGACTTTTTC 3′
3′CGGGCCCGAGCTGAAAAAGAGCT 5′
Transcribed sequence of the h9U6 gene:

(SEQ ID NO:7)

GTCGAGCCCGGGCTCGACTTTTT
h20U6:

(SEQ ID NO:8)

5′TCGAGGATATCGACTGCGCGGGCAGTCGATATCCTCGACTTTTC 3′
3′CCTATAGCTGACGGGCCCGTCAGCTATAGGAGCTGAAAAAGAGCT 5′
Transcribed sequence of the h9U20 gene:

(SEQ ID NO:9)

GTCGAGGATATCGACTGCCCGGGCAGTCGATATCCTCGACTTTTT
nh20U6:

(SEQ ID NO:10)

5′TCGAGGATATCGACTGCCCGGGCAGAGATAAGGTCGACTTTTTC 3′
3′CCTATAGCTGACGGGCCCGTCTCTATTCCAGCTGAAAAAGAGCT 5′

The RNA corresponding to a helix-shaped structure on 18 bases, with a helix interruption on 6 bases. Its sequence is shown below (SEQ ID NO: 11)

GTCGAGGATATCGACTGCCCGGGCAGAGATAAGGTCGACTTTTT

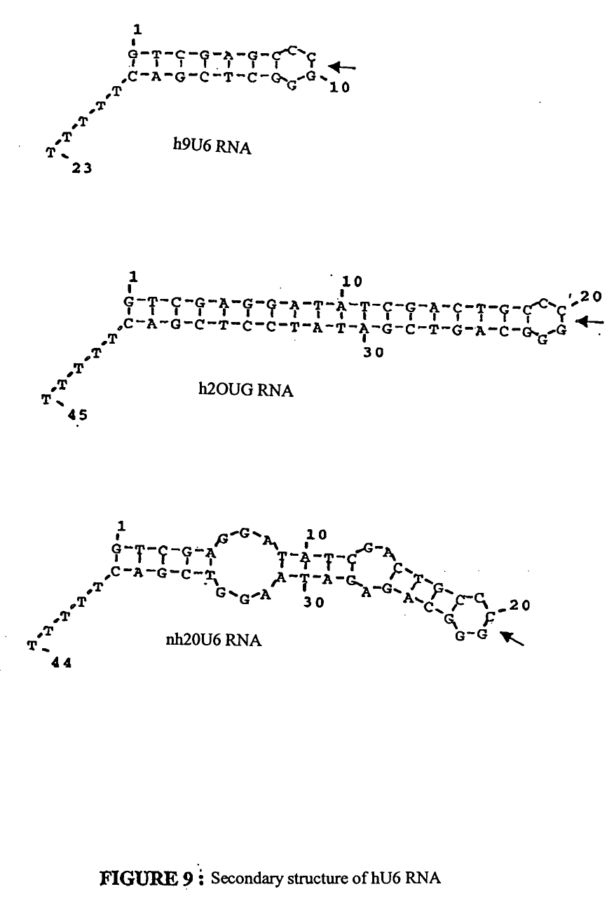

The secondary structure of the U6 helices is shown in FIG. 9.

### A-2-b. Cassette U6 Srf

The aim of this cassette is to express RNA, the sequence of which is chosen from transcription point +1.

In this cassette, the restriction site with Sal I cohesive ends is replaced by the SrfI free edge restriction site.

For this, the U6 promoter is modified by PCR reaction with, as a matrix, the U6 cassette described above. In this reaction, the 5′ primer is a plasmidic sequence located upstream of the promoter, and the 3′ primer makes it possible to modify the sequences close to the +1 transcription site:
5′ GTGGGCCATGGGTGCCCGGGCTTTCGTCCTTTCCACAΔG3′ (SEQ ID NO: 12). In this oligo, the sequence Srf I GCCCGGGC replaces the sequence CACCGTCG present in the original gene (Bertrand et al., 1997) (5° CACCGTCG3′ of the original cassette was changed by the SrfI site 5′ GCCCGGGC 3′ (the underlined sequences represent the start of the transcribed sequences).

The size of the RNA transcribed is a function of the sequence cloned downstream of the promoter inside the SrfI site. In the same way, the cellular localisation of the transcribed RNA is a function of the sequence of the latter.

### A-2-c. Cassette U6Tt

The aim of this cassette is to express active RNA sequences (aptamer, antisense, ribozyme, RNAi: siRNA or miRNA) which are not degraded by the cellular RNAses.

Downstream of the U6 promoter, within said SalI restriction, a sequence called the terminal stem is cloned. It generates a hair-pin shaped structure at the 3′ end of the RNA, thus avoiding degradation by the RNAses. Upstream of this terminal stem is the GTCGAC sequence which restores a SalI restriction site so as to represent the cloning site of the antisense sequences. Downstream of the terminal stem is the transcription stop signal of the polymerase III: TTTTT.

Sequence of the terminal stem (SEQ ID NO: 13)
5′ GCGGACTTCGGTCCGCTTTTT 3′
   The underlined sequences form the RNA helix.

The transcribed sequence in this cassette is (SEQ ID NO: 14)
5′ G//TCGACCCATGCTAGAGCGGACTTCGGTCCGCTTTTT
   //represents the SalI insertion site for the active sequences.
   The sequences in bold print represent the terminal stem.

### A-3. Hybrid Cassettes U6/VA

This example illustrates the structure of a hybrid gene which can in particular be used in murine lines. Indeed, the VA1 gene, as well as the expression cassettes derived from this gene, does not express itself in murine lines. In order to palliate this lack of expression, the U6 murine gene promoter is used to transcribe the expression cassettes derived from the VA1 gene.

The mU6/VAiO structure was implemented in two stages: the first consisted of inserting the U6 promoter of the murine gene (mU6) into the pBabe retroviral vector, and the second consisted of inserting the VAiO gene downstream of the mU6 promoter.

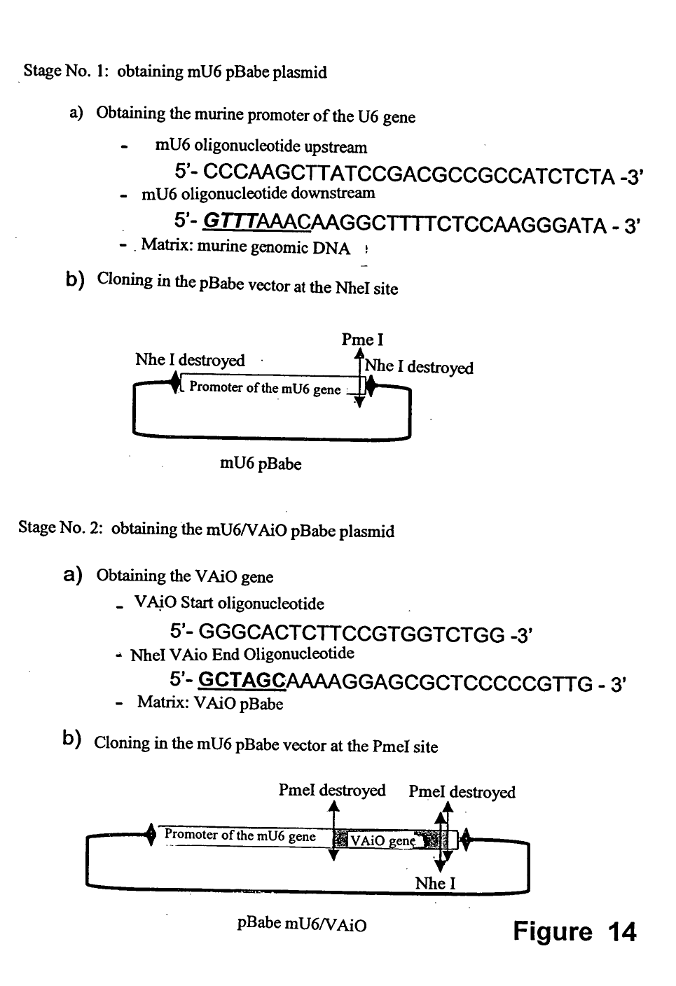

Stage No. 1: Cloning of mU6 in pBabe (FIG. 14)

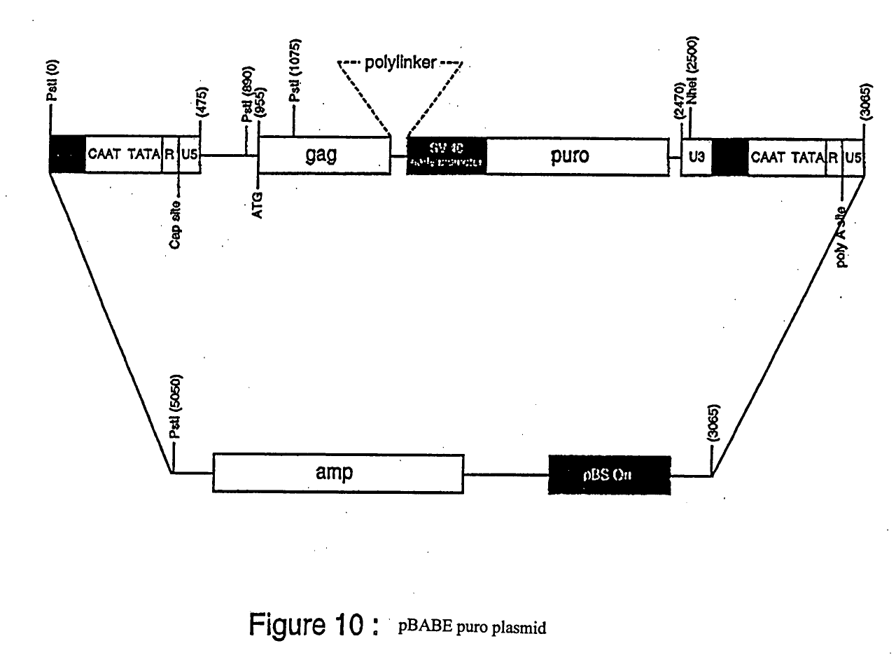

The murine U6 gene was copied by chain polymerisation reaction using mU6 primers upstream and mU6 downstream. In 3′ mU6 primer downstream, 4 extra bases were added so as to integrate a restriction site. The restriction site chosen is the Pme I site of sequence GTTTAAAC. This restriction site has two advantages: it makes it possible i) to keep intact the final bases of the murine U6 promoter (GTTT) and ii) to integrate a free edge cleavage site which can be used in stage No. 2 (insertion of the VAiO gene). The chain polymerisation reaction was implemented with, as its matrix, genomic DNA extracted from murine cells. The reaction product was purified, then inserted into the pBabe retroviral vector at the NheI restriction site (FIG. 10) in order to generate the mU6 pBabe plasmid.

Stage No. 2: Cloning of mU6 in mU6 pBabe (FIG. 14)

The VAiO gene, obtained by chain polymerisation reaction, was cloned in the mU6 pBabe plasmid at the previously introduced PmeI restriction site. The oligonucleotides used to obtain the VaiO gene are VAiO 5′ and VAiO End NheI, an Nhe I restriction site being introduced in 3′ of the NheI End VaiO primer. The DNA matrix used was the VaiO pBabe plasmid.

### B. Cloning of the Expression Cassettes in a Vector

### B-1. p Babe Retroviral Vector

The retroviral vector chosen is the pBabe vector (Morgenstern and Land, 1990). The insertion site chosen is the NheI site which is localised in the 3′ LTR (FIG. 10). This insertion site has several benefits:
During the viral cycle, the 3′ LTR is that which is copied so as to generate the two new LTR of the integrated provirus. It is therefore responsible for the activity of the viral promoter in an integration situation in the cellular DNA. The integration of an exogenous sequence within this LTR does not interrupt the production of recombinant viruses by the encapsidation cells, but makes it possible to inactivate the viral promoter in an integrated situation in the transduced cells. The integration of these sequences into a cellular genome does not therefore involve the activation of the genes located downstream of the insertion. This type of structure is especially beneficial within the context of use for projects relating to gene or cell therapy.
One of the consequences of the duplication of LTR 3′ is also the duplication of the sequence inserted in this LTR. There are therefore two copies of the expression cassettes for each integration.

The cassettes constructed are thus transferred into the pBabe vector at the NheI insertion site (within the 3′ LTR).

### B-2. Other Vectors

Any other vector or means which makes it possible to make the expression cassettes penetrate into the cell is also considered: transfection, transduction or others. Moreover, any vectorisation aiming to make the active RNA, and not the expression cassette penetrate into the cell, is also considered. In particular, any vectors and methods facilitating the introduction of RNA into cells will be used.

### C. Production of Expression Banks of Random RNA

An expression bank of random RNA was generated from fragments of single-stranded DNA with a size of 42 bases. These fragments decompose in three parts: from 5′ to 3′ one finds a known sequence of 8 bases (runA), then a random sequence of 26 bases (chemically synthesized in a perfectly random way with a DNA synthesis machine), and to finish, a second known sequence of 8 bases (runB). The runA and runB sequences are complementary. In a specific example, the single-stranded DNA fragments have the sequence 5′ ATGAACGC (N)26 GCGTTCAT 3′, in which N represents any base (A, T, G or C). The random part therefore contains 26 variable positions.

An oligonucleotide primer complementary to runB was used as a primer for the Kleenow RNA polymerase which synthesises the complementary DNA strand to the whole sequence. In the specific case given in the example, the primer complementary to runB has the sequence 5′ ATGAΔCGC 3′.

At the end of this synthesis stage, one obtains a double-stranded DNA population which has free ends called “bank of random sequences”.
5′-ATGAACGC (N)₂₆ GCGTTCAT-3′| double-stranded DNA: “bank of random sequences”
3′-TACTTGCG (N)₂₆ CGCAAGTA-5′|

This bank of random sequences is then cloned in an expression cassette as described in examples A and B above. According to the type of expression required and/or the needs, the random sequences can be inserted into the two types of cassette derived from VA1 or U6. For this, the plasmids or viral vectors (for example, pBabe in the case of retroviral vectors) containing the expression cassettes are digested by the SrfI restriction enzyme, then purified. The bank of random sequences (double-stranded DNA with free edges) is cloned by a competitive ligation in the presence of the SrfI restriction enzyme. Statistically, each vector therefore integrates a fragment of double-stranded DNA containing a different random sequence. The heterogeneous population of plasmids or vectors obtained after the insertion of random sequences is called “bank of random plasmids or bank of random vectors”. These banks are expression banks of random RNA, in the sense of the invention, directly usable for the in cellulo selection of active RNA.

### D. Production of a Bank of Random Retroviruses

In a preferred embodiment, the expression bank used is a viral expression bank, more particularly retroviral. This type of bank can be constructed from a bank of random viral vectors, as described below.

The bank of random vectors is introduced into the cells of an encapsidation line (here, line 293GP (Bums et al., 1993)) by calcium phosphate transfection. The bank is simultaneously transfected with an expression vector encoding the envelope glycoprotein G of the vesicular stomatitis virus (VSV-G). This protein has the reputation of being very effective for the functionality (stability, infectiosity) of retroviral viruses produced after transfection. A few days after the transfection, the cells produce recombinant pBabe retroviruses. The “Random virus bank” is then collected, purified and concentrated. Its infectiosity level or MOI (number of infectious recombinant retroviral particles per millilitre) can then be determined in accordance with the methods known by experts in the field.

### E. In Cellulo Selection

The expression banks described in examples C and D can be used for the in cellulo selection of active RNA. Thus with the “Random virus bank”, for example, it is possible to transfer the random RNA expression cassettes into the cells to be studied. The aim is to obtain a bank of random cells in which each cell expresses one or more different random RNA. The cells used can be those from a reference line, such as for example cells 293 (ATCC No. CRL 1573), Jurkat A3 cells (ATCC No. CRL-2570) or HELA cells (ATCC No. CCL 2). In another situation, the cells to be infected have a particular nature in the sense that they have a specific activity upon which one wants to act (growth, differenciation, infection . . . ). In all of the cases, one obtains a “random cell bank” which can be kept and perpetuated ad infinitum so as to be used in different applications.

The “bank of random viruses” makes it possible to transduce the cells of any line as well as cells in primary culture. In order to create a “Bank of random, standard or specific cells”, the principle is to obtain a maximum number of transduced cells, each of them having integrated a minimum number of expression cassettes. The aim is to come close to the value of a single random sequence integrated by cellular genome. The cells are thus infected using a viral supernatant with an MOI of less than 1. The selection of the transduced cells is made using the presence of the puromycin resistant gene in the recombinant pBabe retroviruses. After the selection agent has acted, the cell population obtained is therefore made up by all of the cells having received one or more copies of the retroviral genome and therefore containing one or more copies of the pot III expression cassette, each encoding a different random RNA.

The cells of the bank are available for a first series of tests. The target upon which we wish to act by means of the active RNA can be known (a protein, an RNA, a DNA . . . ) or can represent an enzymatic activity, a metabolic path, a proliferation or differenciation process, a resistance to a drug or to a pathogenic agent, etc.

The selection test must be adapted to each case. In a preferred embodiment of the invention, within the bank of random cells, the selection of cells containing an RNA acting upon the target is implemented by the positive selection of cells having acquired the required phenotype and having a selective advantage. In a first example, the selection of an RNA which is active against an infectious agent, such as a cytopathogenic virus (HIV) is implemented by the positive selection of cells which have become resistant to the multiplication of this virus. In another example, the selection of an active RNA capable of protecting the cells against a programmed cellular death process (apoptosis) is implemented by the selection of cells which have become resistant to the addition of a signal which triggers apoptosis. The positive selection of the cells of interest can also include a direct selection of cells by direct observation (overproliferation, change of morphological status, altered differenciation, expression of a fluorescent membrane marker . . . ). The colonies of cells of interest are collected by microdissection with the help of a machine which makes possible the laser microdissection of the groups of cells of interest (Simone et al., 1998) or else by a positive tri based upon the selection of the cells of interest marked with antibodies. After this first “selection circuit”, the cells potentially containing an active RNA are isolated: they make up the first generation of selected cells.

At this stage, the cells selected can be amplified naturally by means of their proliferation.

After sufficient growth, the cells can be cloned in the case where they appear in the form of independent clones, each comprising several thousands of cells. Alternatively, the cells can be globallyjoined together to form a population of cells from the first circuit. In all of the cases, the cells can be conserved by freezing.

This first generation of cells selected (population or independent clones) can be used directly to implement a second selection circuit, then several selection circuits in accordance with an iterative mode, with the possibility of varying the selection parameters.

In certain cases, the selection mode and/or the analysis mode for the cells selected can make it necessary to work on dead cells because they are fixed by a fixing agent such as, for example, formaldehyde. More particularly, this is the case when the selection mode imposes the marking of cells with the help of antibodies. In this case, the natural amplification of the cells by growth is not possible, and an extra stage of amplification by PCR of the cassettes containing active RNA is implemented so as to carry out a second selection circuit.

The genomic DNA of cells coming from the first “selection circuit” is extracted and purified. From this DNA, the techniques of molecular biology make it possible to amplify specifically by PCR the DNA sequences corresponding to the expression cassettes of random RNA contained in the selected cells. One thus obtains a first generation of expression cassettes containing sequences which are potentially active on the target. This first generation is called the “restricted bank of cassettes from the first circuit”.

### F. Selection of Active Cassettes in Accordance with an Iterative

Method

The restricted bank of cassettes from the first circuit is treated in the same way as the random bank from the start. It is cloned in the pBabe retroviral vector at the 3′ LTR. After this new cloning stage, one obtains a first restricted bank of viral vectors. The infectious forms of these viral vectors are obtained identically to above by the transfection of the encapsidation cells in order to end up with a first restricted bank of retroviruses used, in turn, to infect the cellular type being studied. The MOI is adjusted once again so as to be less than 1 virus copy per cell. The cells transduced in this way are selected once again following the same rules as those which govern the selection of the first circuit, or in accordance with other parameters. On thus ends up by establishing a restricted bank of cassettes from the second circuit. This second selection circuit makes it possible to enrich the restricted bank from the first circuit with active sequences.

The iterative succession of circuits based upon the principle of selection for the desired phenotype makes it possible, with each circuit, to enrich the restricted bank with sequences of active cassettes. When the desired phenotype seems to be finally stabilised, ie. when all of the cells having received a recombinant viral vector have the desired phenotype (generally after 5 to 6 selection circuits), it is considered that the selection of cassettes in the cells has been accomplished.

### G. Identification of Active Cassettes and Testing Their Functionality

From these cells selected, preferably during the last selection circuit, the genomic DNA is extracted and purified. An amplification with PCR then makes is possible to obtain the final restricted bank of active cassettes. The cloning of these cassettes in the cloning vectors makes it possible to separate them physically by the propagation of these vectors in bacteria forming isolated colonies. The sequencing of the cassettes found in the different bacterial colonies (generally about thirty colonies are analysed) makes it possible to know the most frequent random sequences or to determine a motif contained in the random sequences which has in particular been conserved during the selection process. Knowing this motif makes it possible to define and to construct one or more cassettes containing this motif by molecular biology.

The cassette or cassettes defined in this way can, at this stage, be analysed individually so as to validate their effectiveness in acting on the target. For this, the cassette is cloned in the pBabe vector, the homogeneous retroviral vectors obtained are then used individually in order to produce recombinant viruses which serve in turn to infect the cells being studied. Comparison of the relative effectiveness of the different cassettes analysed makes it possible to choose the cassette/s which are best adapted to act upon the target.

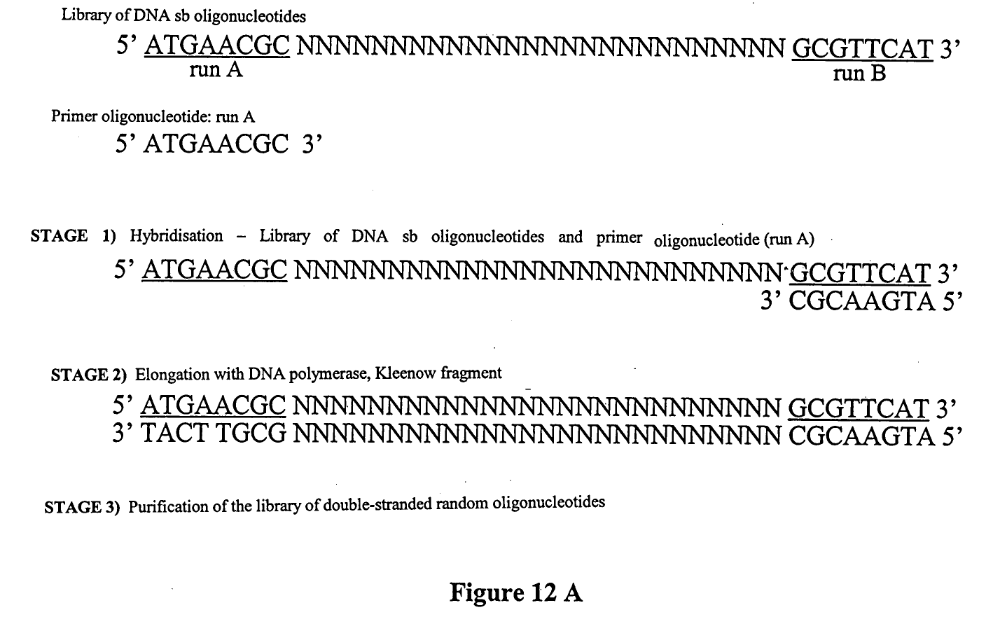

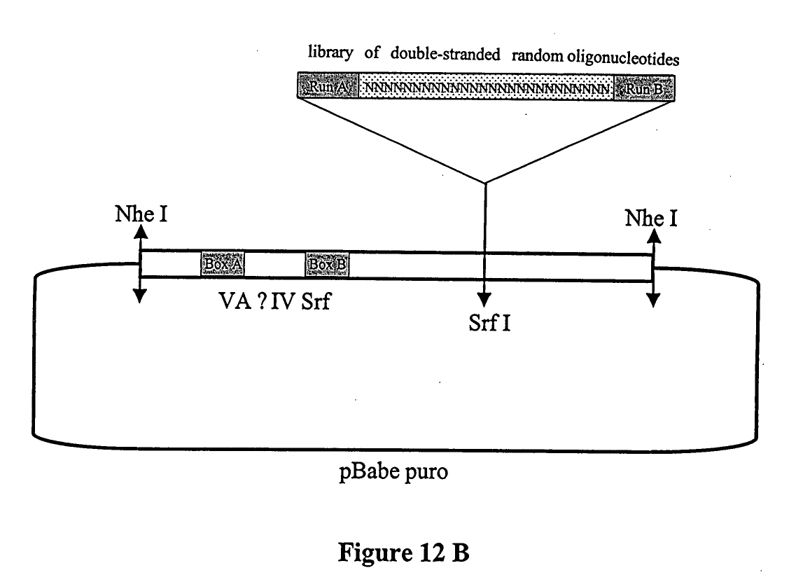

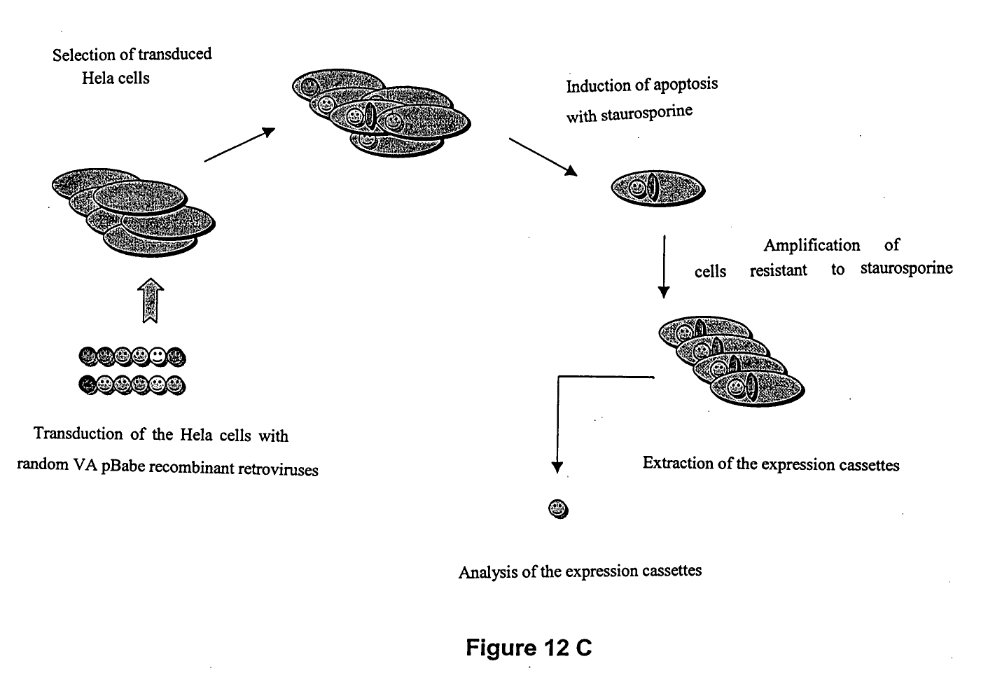

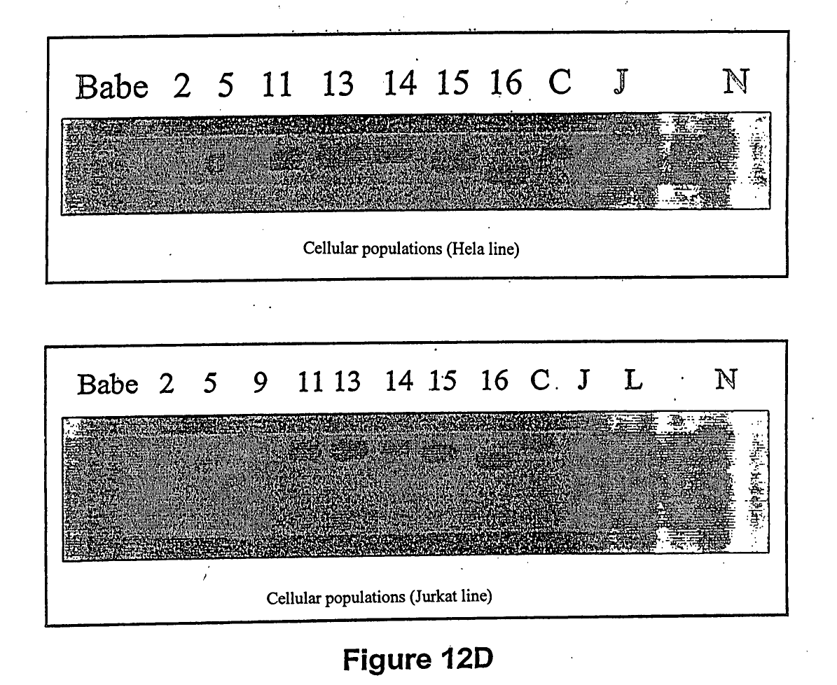

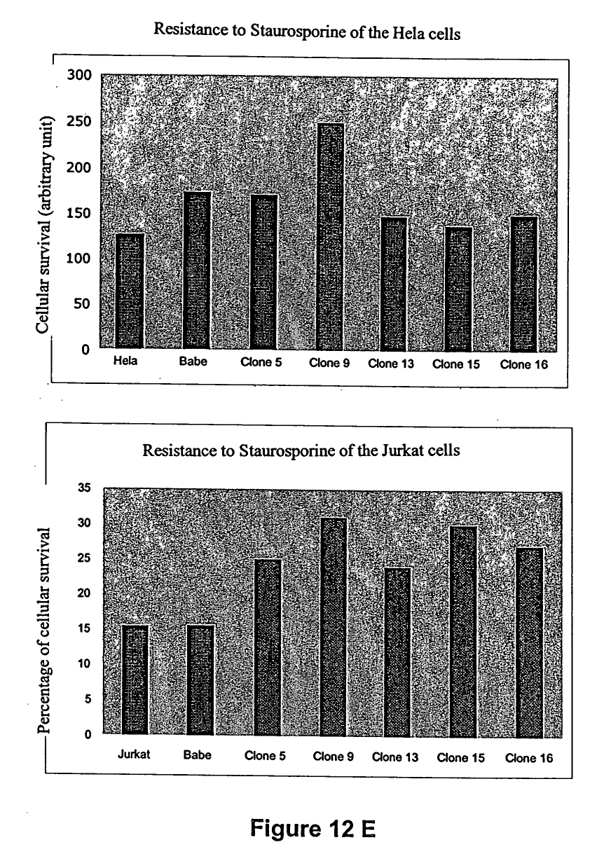

This screening method was used in order to identify the active RNA capable of making the cells from the Hela line resistant to cellular apoptosis induced by staurosporine (0.8 μm-6 hours). At the end of the first selection circuit, different cellular clones which are resistant to staurosporine were selected (FIG. 12C), and the expression cassettes which they contain were identified: clones 2, 5, 9, 11, 13, 14, 15, 16, C, J, L and N. The validation of the active RNA identified in this way was carried out in accordance with the mode indicated above both in the Hela cells and in the Jurkat cells (with an infection multiplicity of less than 1). In each of the cellular populations, the expression level of the active RNA was evaluated by Northern blot (FIG. 12D). In this figure, we can see that the expression level of each of the RNA is comparable from one cellular line to the other (Hela versus Jurkat). New tests for resistance to apoptosis induced by staurosporine were carried out on Hela or Jurkat cell populations, each of them expressing one of the active RNA. Of the 12 RNA studied, only some of them show significant activity resistant to Staurosporine, the others are false positives. The results obtained in these two cellular lines indicate that the resistance rate of the cells to apoptosis varies as a function of the RNA expressed. From the RNA selected, clones 5, 9, 13, 15 and 16 show significant activity in the Jurkat cells (the clone 9 RNA being the most active), whereas only clone 9 shows anti-apoptotic activity in the Hela cells (FIG. 12E). Use of an inducible system such as the VAi system can facilitate the different stages described in the method so as to validate the active RNA directly in the cells in which they were selected (direct elimination of false positives, FIG. 15 Panel A)). With this objective, the VAi system is validated in the Hela T-Rex line cells (invitrogen ref: R714-07) which constitutively express the TetR transgene of the bacterial repressor of the Tetracycline gene (FIG. 7C).

### H—Construction of Libraries of Expression Cassettes Adapted to in Vitro Expression

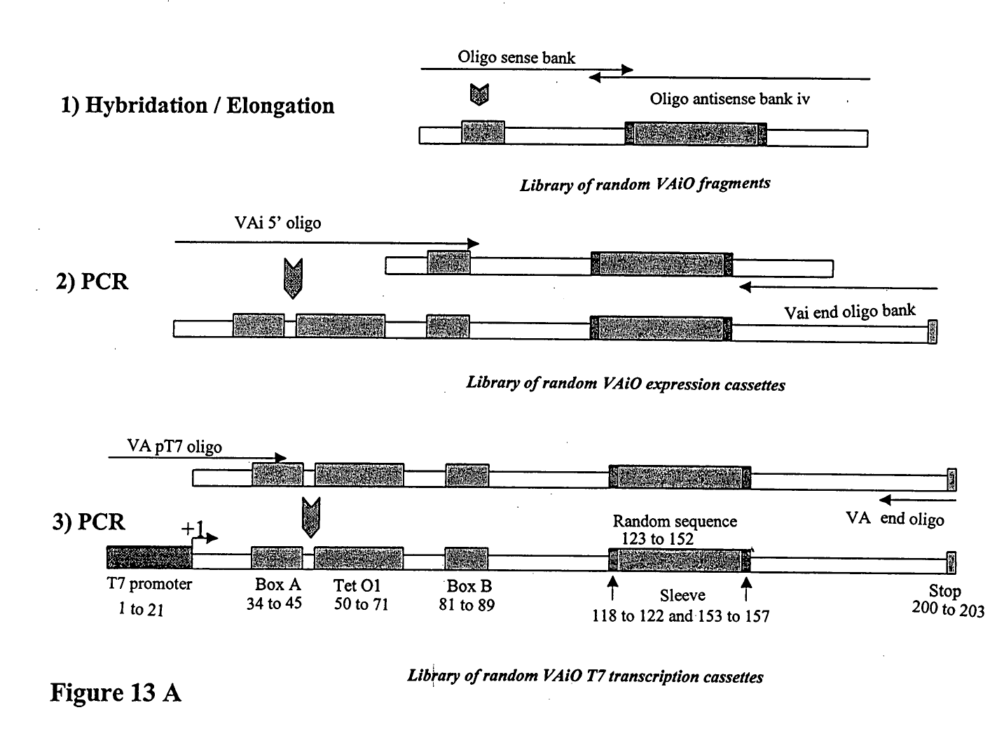

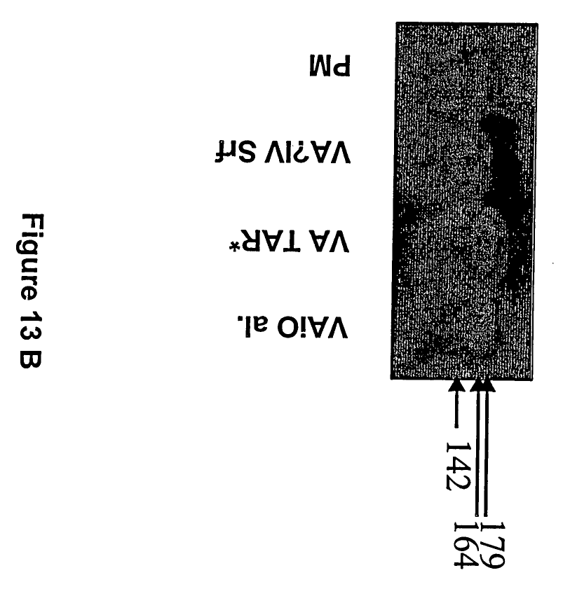

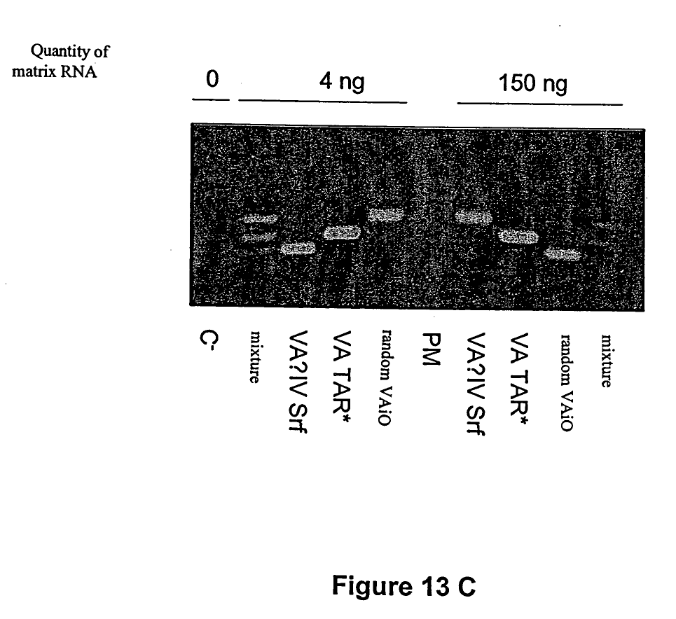

A library of random cassettes was generated in vitro from single-stranded DNA fragment in three stages (FIG. 13A).

The first stage consists of hybridising and elongating two DNA strands so as to recreate a library of fragments of the VA gene into which is inserted a random sequence.

This fragment then serves as the matrix to a chain polymerisation reaction which makes it possible to obtain the library of random VA expression cassettes in its entirety (2^{nd} stage).

The 3^{rd} stage consists of adding, upstream of the random VA expression cassettes, the sequences of a promoter which can be used to carry out in vitro transcription.

From this random VA transcription library, an in vitro transcription stage makes it possible to obtain the random VA RNA library.

In a specific example, the fragments of single-stranded DNA from the first stage are as follows:

sense strand: Sense bank
5′GCGACCGGGGTTCGAACCCCGGAATAACTCTATCAATGATATGCCCAGCCC3′
antisense strand: Antisense bank
5′-GGAACTTCTTGATGCCCTGCCC(N)₃₀3′

in which N represents a random base: A or T or G or C

In this example, the random sequence is represented by a fragment of 30 bases flanked upstream and downstream by two constant sequences of 5 bases capable of forming a double helix structure in the final RNA molecule. This structure is positioned at a level equivalent to the insertion of random sequences into the SrfI site of the VAiO inducible expression cassette (see FIG. 7). After a hybridisation stage, the two oligonucleotides are elongated by the Kleenow fragment DNA polymerase which produces the double-stranded random VAiO fragment (FIG. 13A). The second stage consists of constructing, from the double-stranded random VAiO fragment, the full size library of random VAiO inducible expression cassettes. This stage was implemented by a chain polymerisation reaction with the help of the following oligonucleotides:

sense oligo: VAi5′
5′GGGCACTCTTCCGTGGTCTGGTGGATAAACTCTATCATTGATAGAGTTATGC
GACCGGGG3TCGAACCCCGG 3′
antisense oligo: VAi end bank
5-AAAAGGAGCGCTCCCCCGTTGTCTGACGTCGAACTTCTTGATGCCCTGCCC-
3′

using a Taq DNA polymerase (FIG. 13A). During the third stage, the T7 bacteriophage promoter was added upstream of each random VAiO inducible expression cassette in order to generate the library of random VAiO inducible transcription cassettes. This stage was implemented by chain polymerisation reaction by using oligonucleotides.

VApT7:
5′-AAATTAATACGACTCACTATAGGGGACTCTTCCGTGGTCTGG-3′ upstream
and
VA end:
5′-AAAAGGAGCGCTCCCCCGTTG-3′ downstream.

The library of random VAiO T7 transcription cassettes obtained in this way was used to generate in vitro a library of random VAiO RNA by using T7 bacteriophage RNA polymerase (FIG. 13B). In these experiments, several tens of micrograms of RNA were obtained.

### I—In Vitro Screening of Active RNA and Obtaining Restricted Libraries of Expression Cassettes

The library of random VA RNA is used to select RNA capable of binding a specific substrate in vitro. The active RNA selected by any adapted method known to an expert in the field serve as a matrix for generating the corresponding expression cassettes by a reverse transcription stage followed by an amplification stage (RT-PCR reaction). This mixture of active RNA expression cassettes is then cloned in a vector adapted to the transfer of genes, and then used as a starting material for the in cellulo selection of active RNA (library of random RNA expression cassettes enriched in active RNA).

In a specific example, the VAΔIV Srf, VA TAR* or random VAiO RNA were used in order to obtain the corresponding transcription cassettes. The RT-PCR reaction was implemented in the presence of adapted primers: VApT7 and VAend (FIG. 13C). On the other hand, in order to show that the RT-PCR reaction product is truly representative of the diversity of the substrate, RNA mixtures were used as a matrix. FIG. 13C shows that in the conditions in which three matrix RNA are mixed (VAΔIV Srf RNA, VATAR* RNA and random VAiO RNA) in an equimolecular way (1/3; 1/3; 1/3), the RT-PCR reaction produces three cassettes, the respective quantities of which reflect the initial quantities of each of the substrate RNA. Thus, in the conditions where the random VAiO RNA library is used as a substrate, the RT-PCR reaction products are representative of a library of expression cassettes.

### J—EXPRESSION OF ACTIVE RNA WITH A DETERMINED SEQUENCE

The structures of the invention can also be used for testing defined active sequences, and/or for expressing these sequence in biological tissues.

In this case, the active sequence to be inserted is seen in the form of double-stranded DNA, the sequence of which was chosen in order to generate a effective active RNA (of the antisense, ribozyme, interfering RNA (siRNA, miRNA or their precursors) or aptamer RNA types). Double-stranded DNA can be obtained by different techniques such as hybridisation between two complementary oligonucleotides, the purification of restriction fragments, the copy of a matrix by PCR, etc.

A vector such as that described in example B, containing an expression cassette, is digested with the adapted restriction enzyme, and the cloning of the active sequence takes place in this restriction site by classic cloning techniques.

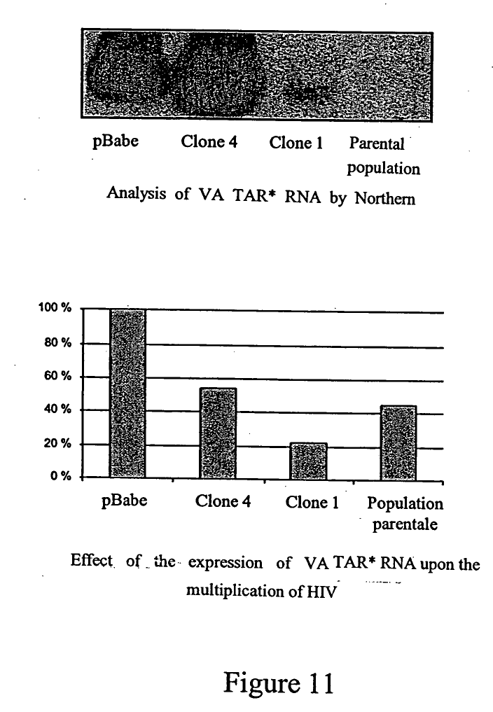

Vectors—whether plasmidic or viral vectors, for example—can be produced in this way. On the other hand, recombinant viruses can also be generated, as described in example C. The recombinant retroviruses produced then serve to infect the cells, the phenotype of which one wants to alter. The infection is preferably implemented with a strong infection multiplicity so as to integrate a high number of active RNA expression cassettes in the cellular genome. Indeed, the activity of the active sequence is strongly dependent upon its expression level (FIG. 11). The presence of the gene resistant to puromycin in the pBabe retroviruses makes possible a rapid selection of the cells which were infected and containing the transduced sequence.

These vectors can also be purified and conditioned in any acceptable vehicle or excipient so as to produce administrable compositions, for example in mammalian organisms, more particularly humans.

### REFERENCES

Barcellini, C. S., Fenard, D., Bertrand, E., Singer, R. H., Lefebvre, J. C., and Doglio, A. (1998). 3′-End modification of the adenoviral VA1 gene affects its expression in human cells: consequences for the design of chimeric VA1 RNA ribozymes [In Process Citation]. Antisense Nucleic Acid Drug Dev 8, 379-90 379-90.
Bertrand, E., Castanotto, D., Zhou, C., Carbonnelle, C., Lee, N. S., Good, P., Chatterdjee, S., Grange, T., Pictet, R., Kohn, D., Engelke, D., and Rossi, J. J. (1997). The expression cassette determines the functional activity of ribozymes in mammalian cells by controlling their intracellular localization. Rna 3, 75-88 75-88.
Bertrand, E., Gwizdek, C., Fenard, D., and Doglio, A. (1999). L'ARN molécule thérapeutique? vers une conception rationnelle du développement des ARN antisens, des ribozymes et des aptaméres d'ARN. Medecine/Sciences 15, 677-681.
Burns, J. C., Friedmann, T., Driever, W., Burrascano, M., and Yee, J. K. (1993). Vesicular stomatitis virus G glycoprotein pseudotyped retroviral vectors: concentration to very high titer and efficient gene transfer into mammalian and nonmammalian cells. Proc Natl Acad Sci USA 90, 8033-7.
Cagnon, L., Cucchiarini, M., Lefebvre, J. C., and Doglio, A. (1995). Protection of a T-cell line from human immunodeficiency virus replication by the stable expression of a short antisense RNA sequence carried by a shuttle RNA molecule. J Acquir Immune Defic Syndr Hum Retrovirol 9, 349-58 349-58.
Charneau, P., Mirambeau, G., Roux, P., Paulous, S., Buc, H., and Clavel, F. (1994). HIV-1 reverse transcription. A termination step at the center of the genome. J Mol Biol 241, 651-62.
Famulok, M., and Verma, S. (2002). In vivo-applied functional RNAs as tools in proteomics and genomics research. Trends Biotechnol 20, 462-6.
Gmeiner, W. H. (2002). The structure and dynamics of the U4/U6 snRNP: implications for pre-mRNA splicing and use as a model system to investigate the RNA-mediated effects of (5F) Ura. J Biomol Struct Dyn 19, 853-62.
Gold, L. (1995). The SELEX process: a surprising source of therapeutic and diagnostic compounds. Harvey Lect 91, 47-57 47-57.
Gossen, M., and Bujard, H. (1992). Tight control of gene expression in mammalian cells bt tetracycline-responsive promoters. Proc. Natl. Acad. Sci. USA 89, 5547-5551.
Gwizdek, C., Bertrand, E., Dargemont, C., Lefebvre, J. C., Blanchard, J. M., Singer, R. H., and Doglio, A. (2001). Terminal minihelix, a novel RNA motif that directs polymerase III transcripts to the cell cytoplasm. Terminal minihelix and RNA export. J Biol Chem 276, 25910-8.
Hermann, T., and Patel, D. J. (2000). Adaptive recognition by nucleic acid aptamers. Science 287, 820-5.
Hillen, W., and Berens, C. (1994). Mechanisms underlying expression of Tn10 encoded tetracycline resistance. Annu Rev Microbiol 48, 345-69.
Mathews, M. B., and Shenk, T. (1991). Adenovirius-associated RNA and translation control. J. Virol. 65, 5657-5662.
McManus, M. T., and Sharp, P. A. (2002). Gene silencing in mammals by small interfering RNAs. Nat Rev Genet 3, 737-47.
Medina, M. F., and Joshi, S. (1999). RNA-polymerase III-driven expression cassettes in human gene therapy. Curr Opin Mol Ther 1, 580-94.
Morgenstern, J. P., and Land, H. (1990). Advanced mammalian gene transfer: high titre retroviral vectors with multiple drug selection markers and a complementary helper-free packaging cell line. Nucleic Acids Res 18, 3587-96.
Paule, M. R., and White, R. J. (2000). Survey and summary: transcription by RNA polymerases I and III. Nucleic Acids Res 28, 1283-98.
Scherr, M., Morgan, M. A., and Eder, M. (2003). Gene Silencing Mediated by Small Interfering RNAs in Mammalian Cells. Curr Med Chem 10, 245-56.
Simone, N. L., Bonner, R. F., Gillespie, J. W., Emmert-Buck, M. R., and Liotta, L. A. (1998). Laser-capture microdissection: opening the microscopic frontier to molecular analysis. Trends Genet 14, 272-6.
Yamamoto, R., Katahira, M., Nishikawa, S., Baba, T., Taira, K., and Kumar, P. K. (2000). A novel RNA motif that binds efficiently and specifically to the Ttat protein of HIV and inhibits the trans-activation by Tat of transcription in vitro and in vivo. Genes Cells 5, 371-88.

1-38. (canceled)

39. A method for the selection, in cellulo, of active RNA capable of conferring on a cell a desired phenotype, the method comprising:
a) providing a bank of nucleic acids comprising a plurality of species of recombinant retroviruses, each species of retrovirus comprising an expression cassette derived from a VA gene of an adenovirus expressing a distinct random structural RNA,
b) contacting said bank or a part thereof with a population of cells under conditions allowing infection of said cells with said recombinant retroviruses,
c) selecting cells having the desired phenotype, and
d) identifying the cassette or cassettes contained in said cell/s or the active RNA that they express.

40. Method of claim 39, wherein the cassette comprises the sequence of an adenovirus VA1 gene deleted of all or a functional part of loop IV, into which the random structural RNA sequence is inserted.

41. Method of claim 39, wherein the terminal double-helix of the VA gene is altered.

42. Method of claim 39, wherein the VA gene comprises a sequence which provides an inducible expression.

43. Method of claim 42, wherein the sequence providing an inducible expression is a sequence which confers sensitivity to a factor working in trans, preferably a binding site of a transcription factor or of a repressor.

44. Method of claim 43, wherein the sequence providing an inducible expression is represented by one or more operating sequences of a regulated bacterial promoter, preferably of the tetracycline promoter.

45. Method of claim 42, wherein the inducible expression cassette is selected from OVAi (SEQ ID NO: 16), VAiO (SEQ ID NO: 15) and OVAiO (SEQ ID NO: 17) cassettes.

46. Method of claim 39, wherein the cassette further comprises a second transcriptional promoter upstream of the VA promoter.

47. Method of claim 39, wherein the active RNA is a structural RNA.

48. Method of claim 39, wherein the active RNA is an interfering or antisense RNA.

49. Method of claim 39, wherein the bank is contacted with the population of cells under conditions allowing the transfer of a restricted number of cassettes per cell, preferably at an MOI of less than 1.

50. Method of claim 39, wherein the bank of nucleic acids is prepared by a method comprising:
the synthesis of a bank of single-stranded DNA comprising a random region flanked by one or two regions having a defined sequence,
the synthesis of a second strand by means of a DNA polymerase and in the presence of a primer complementary to the defined sequence of the first strand, or of a part thereof, in order to produce a bank of double-stranded DNA comprising a random region,
the cloning of the bank of double-stranded DNA in a retroviral vector, under the control of a promoter derived from a VA gene of an adenovirus, and
the transfection of said vector in a packaging cell line, so as to produce a bank of recombinant retroviruses.

51. Method of claim 39, wherein the bank implemented in stage a) is a restricted random bank encoding random RNA pre-selected for one or more particular properties, preferably for their binding capability and/or for their affinity to a target, for the presence of a structural motif, or of a specific sequence.

52. Method of claim 39, wherein the bank implemented in stage a) is a bank of random mutants of a particular target gene.

53. Method of claim 51, comprising:
1a) preparing a bank of recombinant retroviruses comprising an expression cassette comprising a nucleic sequence encoding a random RNA placed under the control of a promoter derived from a VA gene of an adenovirus, the encoded random RNA having been pre-selected in vitro for their capability to bind or for the affinity towards a target of interest,
1b) contacting this bank or a part of this bank with a population of cells under conditions allowing the transfer of nucleic acid into said cells,
2) selecting one or more cells having the desired phenotype, and
3) identifying the cassette or cassettes contained in said cell/s, or the active RNA that they express.

54. Method of claim 53, wherein the bank of recombinant retroviruses encoding pre-selected random RNA is prepared by:
in vitro synthesis of a bank of expression cassettes (dsDNA), each comprising a nucleic sequence encoding a random RNA placed under the control of a promoter derived from a VA gene of an adenovirus, and, optionally, of an additional promoter;
in vitro expression of the bank of cassettes or of a part of the same, to produce in vitro a bank of random RNA;
in vitro selection of RNA for their capability to bind or for their affinity towards a given target; and
production of a restricted bank of recombinant retroviruses comprising RNA expression cassettes thus selected.

55. Method of claim 54, wherein the in vitro expression of the bank of cassettes is performed in two stages, a stage for the production of transcription cassettes and a stage for the transcription thereof.

56. A method for selecting active RNA, the method comprising (i) the in vitro synthesis of a bank of expression cassettes (dsDNA) coding for random RNA under the control of a promoter transcribed by RNA polymerase III derived from a VA gene of an adenovirus, (ii) the in vitro expression of the bank of cassettes or of a part of the same, to produce in vitro a bank of random RNA, and (iii) the in vitro selection of RNA for their binding capability or for their affinity towards a given target.

57. Method of claim 56, further comprising a stage (iv) of production of a restricted bank of recombinant retroviruses comprising expression cassettes of RNA selected during stage (iii).

58. Method of claim 39, wherein the DNA of the expression cassettes of the cells selected is amplified so as to produce a restricted bank, and in that stages b) and c) of the method are repeated at least once with said restricted bank.

59. Method of claim 39, wherein the population of cells comprises animal cells, preferably mammalian, yeast, plants, insect or bacterial cells.

60. Method of claim 39, wherein the desired phenotype is selected from a capacity or an incapacity for growth, apoptosis, differentiation, migration, resistance to a toxic agent, resistance to an infectious agent or metabolic action.

61. Method of claim 39, wherein the desired phenotype is the expression of a biological target.

62. Method of claim 39, wherein the population of cells comprises cells infected by a virus and the desired phenotype is the resistance to said virus.

63. Method of claim 39, wherein the population of cells comprises tumoral cells, and the desired phenotype is the loss of tumorigenicity.

64. Method of claim 39, wherein the population of cells comprises bacterial cells, and the desired phenotype is sensitivity to a toxic agent.

65. Method of claim 42, wherein the activity of the RNA is selected by cultivating the cells in expression-inducing condition and, preferably, further validated by comparing the induced state and the repressed state.

66. A method of claim 39 for selecting active RNA capable of conferring on a cell a desired phenotype, the method comprising:
a) providing a bank of nucleic acids comprising a plurality of species of recombinant retroviruses, each species of retrovirus comprising an expression cassette derived from the sequence of a VA gene of an adenovirus expressing a distinct random RNA,
b) contacting said bank, or part thereof, with a population of cells under conditions allowing infection of said cells with said recombinant retroviruses,
c) selecting cells having the desired phenotype,
d) extracting or amplifying the sequence of cassettes contained in said cells,
e) cloning sequences obtained in step d) in a vector so as to generate a restricted bank, and
f) repeating, at least once, steps b) to d) with said restricted bank.

67. Method of claim 66, in which said bank of nucleic acids from step a) encodes random RNA pre-selected for a particular property, preferably for their capability to bind, in vitro, a target of interest.

68. A method for selecting RNA active on a determined biological target, the method comprising:
a) providing a bank of nucleic acids comprising a plurality of vectors comprising distinct expression cassettes placed under the control of a promoter transcribed by RNA polymerase III, each comprising a nucleic sequence encoding an RNA constrained or pre-defined so as to act upon a given target,
b) contacting said bank, or a part thereof, with a population of cells expressing or containing the biological target, under conditions allowing the transfer of nucleic acid into said cells,
c) selecting cells having the desired phenotype,
d) extracting or amplifying the sequence of cassettes contained in said cells, and optionally,
e) cloning sequences obtained in d) in a vector so as to generate a restricted bank, and
f) repeating, at least once, steps b) and d) with said restricted bank.

69. A bank of nucleic acids, wherein said bank comprises a plurality of species of recombinant retroviruses, each species of retrovirus comprising an expression cassette derived from a VA gene of an adenovirus expressing a distinct random structural RNA.

70. An expression cassette of an RNA, wherein said cassette comprises a sequence encoding said RNA inserted into a promoter derived from a VA gene of an adenovirus, said promoter comprising a sequence conferring an inducible expression.

71. Cassette of claim 70, wherein the terminal double-helix of the VA gene is altered.

72. Cassette of claim 70, wherein the RNA is a random structural RNA or comprises a defined sequence.

73. Cassette of claim 70, wherein the cassette further comprises a second transcriptional promoter, located upstream of the VA promoter.

74. A vector comprising a cassette of claim 70.

75. A cell comprising a cassette of claim 70 or a vector comprising said cassette.

76. A pharmaceutical composition, wherein said composition comprises a cassette of an RNA, wherein said cassette comprises a sequence encoding said RNA inserted into a promoter derived from a VA gene of an adenovirus, said promoter comprising a sequence conferring an inducible expression, a vector comprising said cassette, a cell comprising said cassette or said vector, or an expression cassette of an active RNA identified or produced by a method of claim 39, or an active RNA made up uniquely of the active motif isolated within said cassette.


SEQUENCE LISTING (30 sequences)

SEQ ID NO: 1 - RNA, 160 nt, adenovirus type 2
gggcacucuuccguggucugguggauaaauucgcaaggguaucauggcggacgaccgggguucgaaccccggauccggccguccgccgugauccaugcgguuaccgcccgcgugucgaacccaggugugcgacgucagacaacgggggagcgcuccuuuu

SEQ ID NO: 2 - RNA, 134 nt, Artificial Sequence
  modified VA1 gene
gggcacucuuccguggucugguggauaaauucgcaaggguaucauggcggacgaccgggguucgaaccccggauccggccguccgccgugauauccaggugugcgacgucagacaacgggggagcgcuccuuuu

SEQ ID NO: 3 - RNA, 142 nt, Artificial Sequence
  modified VA1 gene
gggcacucuuccguggucugguggauaaauucgcaaggguaucauggcggacgaccgggguucgaaccccggauccggccguccgccgugaugcccgggcauccaggugugcgacgucagacaacgggggagcgcuccuuuu

SEQ ID NO: 4 - RNA, 141 nt, Artificial Sequence
  modified VA1 gene
gggcacucuuccguggucugguggauaaauucgcaaggguaucauggcggacgaccgggguucgaaccccggauccggccguccgccgugaugcccgggcauccaggugugcgacgucaauaaacgggggagcgcccuuuu

SEQ ID NO: 5 - DNA, 49 bp, Artificial Sequence
  modification of  VA1 gene
gcccgggcatccaggtgtgcgacgtcaataaacgggggagcgccctttt

SEQ ID NO: 6 - DNA, 23 bp, Artificial Sequence
  Artificial sequence h9U6
tcgagcccgggctcgactttttc

SEQ ID NO: 7 - DNA, 23 bp, Artificial Sequence
  Artificial sequence h9U6 primer
gtcgagcccgggctcgacttttt

SEQ ID NO: 8 - DNA, 45 bp, Artificial Sequence
  Artificial sequence h20U6 primer
tcgaggatatcgactgcccgggcagtcgatatcctcgactttttc

SEQ ID NO: 9 - DNA, 45 bp, Artificial Sequence
  Artificial sequence h20U6 primer
gtcgaggatatcgactgcccgggcagtcgatatcctcgacttttt

SEQ ID NO: 10 - DNA, 44 bp, Artificial Sequence
  Artificial sequence nh20U6 primer
tcgaggatatcgactgcccgggcagagataaggtcgactttttc

SEQ ID NO: 11 - DNA, 44 bp, Artificial Sequence
  Artificial sequence nh20U6 primer
gtcgaggatatcgactgcccgggcagagataaggtcgacttttt

SEQ ID NO: 12 - DNA, 39 bp, Artificial Sequence
  Artificial sequence U6 Sfr primer
gtgggccatgggtgcccgggctttcgtcctttccacaag

SEQ ID NO: 13 - DNA, 21 bp, Artificial Sequence
  Artificial sequence U6Tt primer
gcggacttcggtccgcttttt

SEQ ID NO: 14 - DNA, 37 bp, Artificial Sequence
  Artificial sequence U6Tt primer
gtcgacccatgctagagcggacttcggtccgcttttt

SEQ ID NO: 15 - DNA, 142 bp, Artificial Sequence
  VAiO gene
gggcactcttccgtggtctggtggataaactctatcattgatagagttatgcgaccggggttcgaaccccggaataactctatcaatgatatgcccgggcatcaagaagttcgacgtcagacaacgggggagcgctcctttt

SEQ ID NO: 16 - DNA, 191 bp, Artificial Sequence
  OVAi gene
actctatcattgatagagttatagaccgtgcaaaaggagagcctgtaagcgggcactcttccgtggtctggtggataaactctatcattgtagagttatgcgaccggggttcgaaccccggaataactctatcaatgatatgcccgggcatcaagaagttcgacgtcagacaacgggggagcgctcctttt

SEQ ID NO: 17 - DNA, 192 bp, Artificial Sequence
  OVAiO gene
actctatcattgatagagttatagaccgtgcaaaaggagagcctgtaagcgggcactcttccgtggtctggtggataaactctatcattgatagagttatgcgaccggggttcgaaccccggaataactctatcaatgatatgcccgggcatcaagaagttcgacgtcagacaacgggggagcgctcctttt

SEQ ID NO: 18 - DNA, 90 bp, Artificial Sequence
  primer VAi up
ctgtaagcgggcactcttccgtggtctggtggataaactctatcattgatagagttatgcgaccggggttcgaaccccggaataactcta

SEQ ID NO: 19 - DNA, 100 bp, Artificial Sequence
  primer VAi down
gcttggggccttattgagatagttactatacgggcccgtagttcttcaagctgcagtctgttgccccctcgcgaggaaaaccgaaggaaggtccgcgccg

SEQ ID NO: 20 - DNA, 50 bp, Artificial Sequence
  primer VAi PvuII 5′
gtgacagctgagaccgtgcaaaaggagagcctgtaagcgggcactcttcc

SEQ ID NO: 21 - DNA, 72 bp, Artificial Sequence
  primer OVAi PvuII 5′
gtgacagctgactctatcattgatagagttatagaccgtgcaaaaggagagcctgtaagcgggcactcttcc

SEQ ID NO: 22 - DNA, 53 bp, Artificial Sequence
  primer VAi PvuII 3′
ccgaaggaaggtccgcgccgccgacgacgcgatcgaaaaaaccgtcgacagtg

SEQ ID NO: 23 - DNA, 37 bp, Artificial Sequence
  Artificial Sequence sequence TAR primer
acgaagcttgatcccgtatgccggtcgatcgcttcga

SEQ ID NO: 24 - DNA, 51 bp, Artificial Sequence
  Artificial Sequence DNA fragment adenovirus sense strand
gcgaccggggttcgaaccccggaataactctatcaatgatatgcccagccc

SEQ ID NO: 25 - DNA, 52 bp, Artificial Sequence
  Artificial Sequence DNA fragment adenovirus antisense strand
  misc_feature (23)..(52): N = A, T, C or G
cgaacttcttgatgccctgcccnnnnnnnnnnnnnnnnnnnnnnnnnnnnnn

SEQ ID NO: 26 - DNA, 72 bp, Artificial Sequence
  sense oligo  VAi5?
gggcactcttccgtggtctggtggataaactctatcattgatagagttatgcgaccggggttcgaaccccgg

SEQ ID NO: 27 - DNA, 51 bp, Artificial Sequence
  antisens oligo  VAi end bank
aaaaggagcgctcccccgttgtctgacgtcgaacttcttgatgccctgccc

SEQ ID NO: 28 - DNA, 42 bp, Artificial Sequence
  VApT7 oligonucleotides
aaattaatacgactcactatagggcactcttccgtggtctgg

SEQ ID NO: 29 - DNA, 21 bp, Artificial Sequence
  VA end oligonucleotides
aaaaggagcgctcccccgttg

SEQ ID NO: 30 - DNA, 8 bp, Artificial Sequence
  restriction site PmeI
gtttaaac